(19) United States
(12) Patent Application Publication
(10) Pub. No.: US 2003/0180811 A1
(43) Pub. Date: Sep. 25, 2003

(54) Immunoassays for beta2-microglobulin

(76) Inventors: Felix A. Montero-Julian, Marseille (FR); Antje Necker, La Ciotat (FR)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/096,081

(22) Filed: Mar. 11, 2002

(51) Int. Cl. G01N 33/53; G01N 33/567; G01N 33/537; G01N 33/543; C07K 14/74

(52) U.S. Cl. 435/7.2; 530/350

(57) ABSTRACT

Immunoassays useful for detecting free β2-microglobulin in a sample containing β2-microglobulin/β2-microglobulin associated protein complexes are provided. Also provided are a sandwich immunoassay and a competition immunoassay for detecting free β2-microglobulin in a sample containing MHC monomers or MHC tetramers. Kits for performing such immunoassays also are provided.

### BACKGROUND OF THE INVENTION

### FIELD OF THE INVENTION

The invention relates generally to immunoassays, and more specifically to methods for detecting free β2-microglobulin in a sample, and to kits useful for performing such methods.

### BACKGROUND INFORMATION

The vertebrate immune response includes two arms—the humoral immune response, characterized primarily by the stimulation of B lymphocytes (B cells) to produce antibodies, and the cellular immune response, characterized primarily by the activation of effector T lymphocytes (T cells), including cytotoxic T cells (CTLs), which can “kill” infecting organisms, and helper T cells, which contribute to the stimulation of antibody producing B cells. The humoral and cellular immune responses generally work together in response to an infection, though the humoral immunity generally is activated in response to exposure to a toxin such as a bacterial lipopolysaccharide endotoxin, whereas the cellular immune response generally is activated in response to a viral or bacterial infection or to exposure to a non-self antigen, for example, due to tissue transplantation.

Since the magnitude of an immune response can assist clinicians in following the progression and determining the prognosis of an infection, a great deal of effort has gone into developing methods for measuring the level and persistence of an immune response. For a humoral response, simple methods are available for determining the circulating levels of antibodies in the serum. Methods for measuring the magnitude of a cellular immune response, however, are as straight-forward, since they generally require identifying the T cells involved in the response.

A common method for determining the number of T cells in an individual that are responsive for a particular antigen is the limiting dilution assay. In this method, CTLs are serially diluted in microtiter plates until a single cell on average is present in a well, then the cells are stimulated to proliferate, and examined for cytotoxic activity in response to antigen. This method is useful because it indicates not only that the CTLs have cytotoxic activity, but also that the CTLs can proliferate, which can be critical upon subsequent infection. Unfortunately, the limiting dilution method is time consuming because the cells generally need to proliferate for a couple of weeks such that a sufficient number is present to measure cytotoxic activity. As such, the method is labor intensive and expensive to perform, and is not readily adaptable to a high throughput assay format. In addition, the limiting dilution assay may underestimate the number of specific CTLs in an individual because the method only identifies CTLs that have the capacity to proliferate.

Another method that has been useful for identifying antigen-specific CTLs relies on the expression of cytokines such as interferon gamma by antigen stimulated CTLs. In this method, antigen stimulated cells are permeabilized, and intracellular immunostaining is performed using, for example, detectably labeled anti-interferon gamma antibodies. This method has advantages over the limiting dilution method in that there is no requirement for cell proliferation or, therefore, for a cell culturing step, and it can be readily adapted to a high throughput assay format. However, the method is toxic to the cells and, therefore, it is not possible to select the antigen-specific cells, for example, to perform additional functional tests.

more recently developed method of detecting antigen-specific T cells utilizing tetramers of major histocompatibility complex (MHC) molecules has revolutionized T cell analysis. MHC tetramer complexes are formed by the association of four MHC monomers, for example, four MHC class I molecule/β2-microglobulin monomers, with a specific peptide antigen and a detectable label such as a fluorochrome. Such MHC class I molecule tetramer complexes bind to a distinct set of T cell receptors on a subset of CD8+ T cells, including cytotoxic T lymphocytes (CTLs). CTLs, which are effector CD8+ T cells, do not necessarily represent the whole antigen-specific pool of CD8+ T cells. In this respect, the LDA and cytokine assay both detect CTLs or subpopulations of CTLs, whereas the MHC tetramer method can detect all antigen-specific CD8+ T cells, including naive and anergic CD8+ T cells, which do not exhibit effector functions. By mixing the MHC tetramers with peripheral blood lymphocytes or whole blood, and using flow cytometry as a detection system, a count of all T cells that are specific for a peptide and its matched allele is provided. As such, the MHC tetramers allow for the measurement of a cellular response against a specific peptide.

The use of MHC tetramers to analyze T cell specificity provides significant advantages over previously used T cell assays. For example, the MHC tetramer method is quantitative, it does not require the use of radioactive dyes, and it is readily adapted to high throughput assay formats. In addition, the method can be performed quickly and, therefore, can be used to examine fresh blood or tissue samples. Where the MHC tetramer complex includes a fluorescent label, a cell population including T cells can be further stained with one or more other fluorescently labeled molecules that, for example, are specific for other cell surface molecules and analyzed using flow cytometry, thus allowing additional characterization of the responding cells. Furthermore, MHC tetramer analysis is not toxic to the labeled cells and, therefore, tetramer binding cells can be sorted into uniform populations by flow cytometry and examined by additional assays to confirm their functional ability, for example, the ability to proliferate in response to antigen.

The use of MHC tetramer analysis allows the identification of individual T cells on the basis of the specificity of the binding to the MHC-peptide complex. The tetramer analysis method has been used to study CD8+ T cell responses in humans with acute viral infections such as HIV, where it revealed that the increase of antigen-specific CD8+ T cells during the acute phase of the response was far greater than previously thought. MHC tetramers also have been used to accurately and efficiently monitor CD8+ T cell responses in other viral infections, including Epstein Barr virus-mononucleosis, cytomegalovirus, human papilloma virus, hepatitis B, hepatitis C, influenza and measles; in a parasitic infection, malaria; in cancers, including breast, prostate, melanoma, colon, lung, and cervical cancers; in autoimmune diseases, including multiple sclerosis and rheumatoid arthritis; and transplantation.

The specificity of MHC tetramer binding depends on the intactness of the whole complex (heavy chain, β2-microglobulin, and peptide). The association of heavy chain, light chain and peptide is reversible and depends on the affinity of the peptide for the MHC allele. Dissociation of the complex can be measured by measuring the dissociation product of one of its components, β2-microglobulin (light chain). However, in order to be useful for measuring dissociation, free β2-microglobulin must be measured in a manner that distinguishes it from β2-microglobulin that is bound in a complex. Where the MHC tetramers are to be used as reagents for clinical or other such assays, it is critical that the amount of functional MHC tetramers in the reagent be known, or easily determined so that the assays can be standardized and provide accurate and precise results.

The dissociation of β2-microglobulin from MHC tetramers (and from MHC monomers) generally has been measured by size exclusion chromatography, wherein the amount of free β2-microglobulin is determined in a sample containing the tetramers (or monomers). Essentially, the sample is passed over an appropriate column, fractions eluting from the column are monitored, for example, by UV absorbance, and the area under a peak corresponding to the elution time of free β2-microglobulin is determined using an appropriate algorithm. Unfortunately, the size exclusion method for determining free β2-microglobulin has several shortcomings. For example, while analysis of a single sample can be performed in about 1 to 2 hours, the analysis of a number of samples, including analysis of doublets, must be performed serially. As such, it can take a very long time to analyze a large number of samples. In addition, the variability of the method is rather high, resulting in a relatively imprecise assay. Thus, a need exists for a convenient and efficient method of determining the amount of free β2-microglobulin in a sample containing MHC class I molecule/β2-microglobulin complexes. The present invention satisfies this need and provides additional advantages.

### SUMMARY OF THE INVENTION

The present invention relates to a method of detecting the presence of free β2-microglobulin in a sample that contains complexes of β2-microglobulin and a β2-microglobulin associated protein (β2m-AP). A method of the invention can be performed, for example, by contacting the sample with an antibody, or an antigen binding fragment thereof, that specifically binds free β2-microglobulin, but does not substantially bind β2-microglobulin when it is present as a β2-microglobulin/β2m-AP complex, under conditions that allow specific binding of the antibody, or antigen binding fragment thereof, and β2-microglobulin; and detecting specific binding of the antibody to β2-microglobulin, thereby detecting the presence of free β2-microglobulin in the sample.

The β2-microglobulin can be from any organism, particularly a vertebrate organism, including a mammalian β2-microglobulin such as human β2-microglobulin or murine β2-microglobulin. The β2m-AP can be any molecule that specifically associates with a β2-microglobulin polypeptide, for example, a major histocompatibility complex (MHC) class I molecule. The MHC class I molecule can be MHC class Ia molecule, for example, a murine H2 molecule such as an H2-D, H2-K or H2-L molecule, or a human lymphocyte antigen (HLA) molecule such as an HLA-A, HLA-B, or HLA-C molecule, or can be an MHC class Ib molecule such as an HLA-E, HLA-F or HLA-G molecule. The β2m-AP also can be, for example, the hemochromatosis gene product, HFE, which is involved in iron metabolism, or a cluster of differentiation (CD) molecule such as a CD1a, CD1b, CD1dc CD1d and CD1e molecule.

An antibody useful in a method of the invention specifically binds free β2-microglobulin, but does not bind β2-microglobulin that is in a complex with a β2m-AP, for example, with an MHC class I molecule. Such an antibody is exemplified by the monoclonal antibody C21.48A, and can be an antibody having substantially the same specific binding activity of C21.48A. An antigen binding fragment of such an antibody also can be used in a method of the invention, as can an antibody derived from such an antibody, for example, a single chain antibody. If desired, the antibody can be immobilized to a solid support, which is generally insoluble under the conditions used for performing an immunoassay of the invention.

Detecting specific binding of the antibody to β2-microglobulin can be quantitative or qualitative, and can be performed in various ways. In one embodiment, specific binding of the antibody that specifically binds free β2-microglobulin, but does not substantially bind β2-microglobulin when it is complexed with a β2m-AP (also referred to herein as a “first antibody”), and free β2-microglobulin is detected using a second antibody. In one aspect of this embodiment, specific binding of the first antibody to β2-microglobulin is detected by further contacting the sample and first antibody with a second antibody that specifically binds β2-microglobulin, including β2-microglobulin that is specifically bound to the first antibody, under conditions that allow specific binding of the second antibody; isolating the first antibody, including free β2-microglobulin specifically bound to the first antibody and second antibody specifically bound thereto; and detecting second antibody associated with the isolated first antibody and free β2-microglobulin.

In another aspect of this embodiment, specific binding of a first antibody, which can be an immobilized antibody, to free β2-microglobulin is detected by isolating the first antibody, including free β2-microglobulin specifically bound thereto, from material not specifically bound to the first antibody; further contacting the isolated first antibody, including any free β2-microglobulin specifically bound thereto, with a second antibody that specifically binds β2-microglobulin, including β2-microglobulin that is specifically bound to the first antibody, under conditions that allow specific binding of the second antibody; and detecting second antibody associated with the isolated first antibody/free β2-microglobulin complex.

A second antibody useful in such a method of the invention can be any antibody that specifically binds free β2-microglobulin, or that specifically binds free β2-microglobulin and β2-microglobulin when it is complexed with a β2m-AP, provided the second antibody also specifically binds β2-microglobulin when the latter is specifically bound by a first antibody having the above-described characteristics. For example, the second antibody can be the monoclonal antibody B1G6, which specifically binds to β2-microglobulin regardless of whether it is in a free form or is in a complex with a β2m-AP, or can be an antibody having substantially the same specific binding activity of B1G6.

A second antibody can include a detectable label, for example, a fluorescent molecule, radionuclide, luminescent molecule, or the like, in which case detecting second antibody can be accomplished by detecting the detectable label. In addition, or alternatively, detecting second antibody specifically bound to a first antibody and β2-microglobulin can be performed by contacting the second antibody with a reagent that specifically binds to the second antibody, under conditions that allow specific binding of the reagent to the second antibody, and detecting specific binding of the reagent to the second antibody. Such a reagent can be a third antibody, an Fc receptor, or any other reagent that specifically binds the second antibody or a moiety linked thereto.

In another embodiment of a method of the invention, specific binding of free β2-microglobulin to an antibody that specifically binds free β2-microglobulin, but does not substantially bind β2-microglobulin when it is present as a β2-microglobulin/β2m-AP complex (i.e., a “first antibody”), is detected in the presence of a competitor β2-microglobulin. Such a method is performed, for example, by contacting the sample, the first antibody, and the competitor β2-microglobulin; and detecting competitor β2-microglobulin specifically bound to the antibody, wherein the amount of competitor β2-microglobulin binding is indicative of free β2-microglobulin in the sample. The competitor β2-microglobulin can contain a detectable label, for example, a fluorescent molecule, a luminescent molecule, a radionuclide, or an enzyme such as alkaline phosphatase, or can comprise any means that facilitates detection of the competitor β2-microglobulin.

In a method of the invention, the first antibody can be immobilized or capable of being immobilized to a solid support, thereby facilitating isolating the first antibody, including, where present, free β2-microglobulin or competitor β2-microglobulin specifically bound thereto and, where present, specifically bound second antibody. The first antibody can be immobilized prior to contacting it with the sample; during the time it is contacted with the sample, including, where appropriate, during the time a competitor β2-microglobulin or a second antibody is contacted with the first antibody and sample, or just prior to detecting specific binding, provided that when the immobilization of the first antibody is performed during or after a contacting step, the immobilization does not affect a specific binding interaction.

The present invention also relates to a method of detecting the presence of free β2-microglobulin, which is not bound to an MHC class I molecule, in a sample containing β2-microglobulin/MHC class I molecule complexes. Such a method can be performed, for example, by contacting the sample with an antibody, or antigen binding fragment thereof, that specifically binds free β2-microglobulin, but does not substantially bind a β2-microglobulin/MHC class I molecule complex, under conditions that allow specific binding of the antibody and β2-microglobulin; and detecting specific binding of the antibody to free β2-microglobulin. In one embodiment, the antibody is immobilized on a solid support, thereby facilitating detecting specific binding of the antibody to free β2-microglobulin.

The β2-microglobulin can be from any organism, including a mammalian β2-microglobulin such as human β2-microglobulin or murine β2-microglobulin, and the β2-microglobulin/MHC class I molecule complex can be any complex, including a β2-microglobulin/MHC class I molecule monomer, which include one β2-microglobulin light chain specifically associated with one MHC class I molecule heavy chain; a β2-microglobulin/MHC class I molecule polymer, which includes at least two operatively associated β2-microglobulin/MHC class I molecule monomers; or a combination of monomers and polymers. In one embodiment, the β2-microglobulin/MHC class I molecule polymer is an MHC tetramer. In another embodiment, one or more of the MHC class I molecules in a polymer contains a linker moiety, which facilitates formation of an MHC class I molecule polymer from monomers, including at least one monomer containing the linker moiety.

The linker moiety can be any molecule or molecules that facilitate an association of two or more MHC monomers that is stable under the conditions to which an MHC polymer comprising the linked monomers is to be exposed and that does not disrupt the function of the MHC monomers comprising the MHC polymer, particularly the ability of an MHC monomer to specifically bind a peptide antigen. A linker moiety can be, for example, a thiol reactive group, such that the monomers are operatively linked through a disulfide bond; or can be members of a specific binding pair such that a monomer containing a first member of a specific binding pair is operatively linked to a monomer containing a second member of the specific binding pair, which interacts specifically with a first member of the specific binding pair, or such that two or more monomers, each of which contains a first member of a specific binding pair, is operatively linked to each other through the second member of the specific binding pair. For example, β2-microglobulin/MHC class I molecule complexes in a sample can be β2-microglobulin/MHC class I molecule polymers, wherein each monomer in a polymer is operatively linked through a specific interaction of the first member of a specific binding pair such as biotin, to a second member of the specific binding pair, for example, avidin or streptavidin. The MHC class I molecule monomers, including monomers in an MHC polymer, generally contain a peptide antigen binding domain and can further contain a peptide antigen specifically bound thereto.

The present invention also relates to kits that contain one or more reagents useful for detecting the presence of free β2-microglobulin in a sample that contains complexes of β2-microglobulin and a β2m-AP, for example, MHC monomers, dimers, trimers, tetramers, and the like. In one embodiment, a kit of the invention contains an antibody, or antigen binding fragment thereof, that specifically binds free β2-microglobulin, but does not substantially bind a β2-microglobulin/MHC class I molecule complex, and also can contain a competitor β2-microglobulin, which can be specifically bound by the antibody, or antigen binding fragment thereof. The antibody can be any antibody having the required specificity, for example, the monoclonal antibody C21.48A or an antibody having substantially the same specific binding activity of C21.48A.

The antibody, or antigen binding fragment thereof, in the kit can be immobilized on a solid support, or can be in a form that can be immobilized to a solid support, in which case the kit can further contain reagents for performing the immobilization, including, if desired, one or a few types of the solid supports, to which the antibody can be immobilized. The competitor β2-microglobulin of the kit can be detectably labeled, for example, with a fluorescent molecule, a luminescent molecule, a radionuclide, or an enzyme such as alkaline phosphatase, or can be in a form that is readily labeled, in which case the kit can further contain reagents for labeling the competitor β2-microglobulin. The kit also can contain at least one standard, for example, one or a few predetermined amounts or concentrations of free β2-microglobulin, thus providing a kit useful for quantitating an amount of free β2-microglobulin in a sample.

In another embodiment, a kit of the invention contains a first antibody, or antigen binding fragment thereof, which specifically binds free β2-microglobulin, but does not substantially bind a β2-microglobulin/MHC class I molecule complex, and also can contain a second antibody, which specifically binds free β2-microglobulin, or free β2-microglobulin and β2-microglobulin complexed with an MHC class I molecule, and further specifically binds free β2-microglobulin complexed with the first antibody. For example, the first antibody can be the C21.48A antibody or an antibody having substantially the same specific binding activity of the C21.48A antibody, and the second antibody can be the B1G6 antibody or an antibody having substantially the same specific binding activity of the B1G6 antibody.

The first antibody or second antibody of a kit of the invention can be immobilized to a solid support, or in a form that can be readily immobilized to solid support, in which case the kit can further contain reagents for performing the immobilization, including, if desired, one or a few types of the solid supports. The second antibody of the kit can be detectably labeled, or can be capable of being detected, for example, using a third antibody or other reagent that specifically binds the second antibody or a moiety linked thereto, including at least when the second antibody is specifically bound to β2-microglobulin that is specifically bound to the first antibody. The kit also can contain free β2-microglobulin, β2-microglobulin that is complexed with an MHC class I molecule, or a combination thereof, and when the kit contains free β2-microglobulin, the free β2-microglobulin can be in one or a few predetermined amounts or concentrations, which can be useful as a standard.

### BRIEF DESCRIPTION OF THE DRAWINGS

FIG. 1 shows a dose response curve obtained following incubation of anti-β2-microglobulin antibodies (C21.48A mAb; B1G6 mAb) or an irrelevant antibody (TR10mAb; control) on plates coated with β2-microglobulin.

FIG. 2 shows the results of an ELISA calibration assay. Serial ten-fold dilutions of β2-microglobulin and peroxidase-labeled B1G6 mAb were examined.

FIG. 3 shows an ELISA standard curve. Serial two-fold dilutions of recombinant β2-microglobulin were assayed. Equation of the curve after linear regression is inserted in the Figure. A linear regression curve also was added.

FIG. 4 shows a standard curve of β2-microglobulin assayed according the competition assay method described in Table XI.

FIG. 5 provides a comparison between the level of free β2-microglobulin in MHC tetramer samples assayed by size exclusion chromatography, ELISA, or the competition assay.

FIG. 6 shows an analysis of the results obtained using the enzyme immunoassay or size exclusion chromatography with least squares and Deming linear regression.

### DETAILED DESCRIPTION OF THE INVENTION

The present invention provides immunoassay methods for detecting and quantitating free β2-microglobulin in a sample containing or suspected of containing complexes of β2-microglobulin and a β2-microglobulin associated protein (β2m-AP) such as an MHC class I molecule. Kits for performing such methods also are provided. As disclosed herein, the immunoassay methods of the invention are robust, accurate, sensitive, and reproducible (see Examples 2 and 3).

The present invention provides a method of detecting the presence of free β2-microglobulin in a sample that contains complexes of β2-microglobulin and a β2m-AP. A method of the invention can be performed, for example, by contacting the sample with an antibody, or an antigen binding fragment thereof, that specifically binds free β2-microglobulin, but does not substantially bind β2-microglobulin when it is present as a β2-microglobulin/β2m-AP complex, under conditions that allow specific binding of the antibody, and β2-microglobulin; and detecting specific binding of the antibody to β2-microglobulin, thereby detecting the presence of free β2-microglobulin in the sample. For convenience of discussion, the antibody, or antigen binding fragment thereof, that specifically binds free β2-microglobulin, but does not substantially bind β2-microglobulin when it is present as a complex with a β2m-AP, is referred to generally herein as a “first antibody.”

According to a method of the invention, specific binding of the first antibody to free β2-microglobulin can be detected using a second antibody, for example, in a sandwich assay format such as an enzyme-linked immunosorption assay (ELISA), or can be detected using competitor β2-microglobulin in a competition assay. Where a second antibody is used for detecting specific binding of the first antibody and free β2-microglobulin, the second antibody is selected based on the ability to specifically bind β2-microglobulin that is specifically bound by the first antibody, i.e., a first antibody/β2-microglobulin complex, and can further be selected based on the ability to specifically bind free β2-microglobulin, or to specifically bind free β2-microglobulin as well as β2-microglobulin that is complexed with a β2m-AP.

As used herein, the term “β2-microglobulin associated protein” or “β2m-AP” means a molecule that specifically associates with β2-microglobulin. A β2m-AP is exemplified herein by major histocompatibility complex (MHC) class I molecules, including class Ia molecules and class Ib molecules. MHC class Ia molecules are exemplified by murine H2 molecules such as an H2-D, H2-K and H2-L molecule, and human lymphocyte antigen (HLA) molecules such as an HLA-A, HLA-B and HLA-C molecule, and MHC class Ib molecules are exemplified by HLA-E, HLA-F and HLA-G molecule. The β2m-AP also can be, for example, the hemochromatosis gene product, HFE, which is involved in iron metabolism, or a cluster of differentiation (CD) molecule such as a CD1a, CD1b, CD1c CD1d and CD1e molecule. Generally, an antibody that specifically associates with β2-microglobulin is not considered a β2m-AP for purposes of the present discussion. However, the disclosed methods can be readily used for detecting free β2-microglobulin in a sample containing β2-microglobulin and an anti-β2-microglobulin antibody.

As used herein, the term “complex” is used broadly to refer to any two molecules, particularly proteins, that specifically associate with each other under physiological condition. The term “complex” also includes a specific association of two or more molecular complexes. In particular, the term “complex” is used herein to refer to an association of β2-microglobulin and a β2m-AP, particularly a complex containing an MHC class I molecule associated with β2-microglobulin, and also is used to refer to an association formed between a protein such as β2-microglobulin and an antibody that specifically binds to the β2-microglobulin. The term “MHC monomer” is used more specifically herein to refer to a complex formed between and MHC class I molecule and β2-microglobulin, and the term “MHC polymer” is used herein to refer to a complex containing two or more MHC monomers. An MHC polymer can comprise an MHC dimer, MHC trimer, MHC tetramer, and the like (see, for example, U.S. Pat. No. 5,635,363, which is incorporated herein by reference). The monomers in an MHC polymer can be linked directly, for example, through a disulfide bond, or indirectly, for example, through a specific binding pair, and also can be associated through a specific interaction between secondary or tertiary structures of the monomers such as a leucine zipper, which can be engineered, for example, into a MHC class I molecule component of the monomers. An MHC polymer also can contain a peptide antigen, which generally is specifically bound to the peptide binding site (cleft) of an MHC class I molecule; can further contain a peptide sequence engineered into the class I component of one or more MHC monomers in the polymer, for example, a signal sequence containing a biotinylation site for the BirA enzyme; and can contain a detectable label.

The methods of the invention are useful for detecting the presence or amount of free β2-microglobulin in a sample, including a sample that contains or is suspected of containing a complex comprising β2-microglobulin. As such, a method of the invention can be used, for example, to follow the formation of β2-microglobulin/MHC class I molecule complexes in a reaction designed to form such molecules, thus providing a means for determining the extent of such a reaction and the time the reaction has reached completion or a steady-state; or can be used to determine dissociation of such a complex with time, including in a sample known to contain a particular amount of the complex at a specified time.

The methods of the invention are particularly useful for detecting dissociation of β2-microglobulin from MHC tetramers. MHC tetramers are complexes of four MHC monomers, which are associated with a specific peptide antigen and contain a fluorochrome (U.S. Pat. No. 5,635,363). MHC class I monomers are composed of two polypeptides, an MHC-encoded polymorphic transmembrane polypeptide having a molecular mass of about 45,000 Daltons (Da) and a non-polymorphic β2-microglobulin polypeptide having a molecular mass of about 12,000 Da. The heavy chain includes, from N-terminus to C-terminus, three extracellular domains, designated α1, α2 and α3, a transmembrane domain, and a small cytoplasmic domain; β2-microglobulin associates with the α3 domain. MHC class I monomers have been prepared by substituting the transmembrane and cytoplasmic domains of the heavy chain with a peptide sequence that can be biotinylated, and MHC class I tetramers have been formed by contacting such monomers with streptavidin, which can bind four biotin moieties (see, for example, Altman et al., Science 274:94-96, 1996; Ogg and McMichael, Curr. Opin. Immunol. 10:393-396, 1998, each of which is incorporated herein by reference; see, also, U.S. Pat. No. 5,635,363), and are commercially available (Immunomics/Beckman Coulter, Inc.).

MHC tetramers have been prepared using MHC class I and class II molecules, including mutated class Ia HLA molecules, including HLA-A*0201, HLA-B*3501, HLA-A*1101, HLA-B*0801, and HLA-B*2705 to minimize binding of the HLA molecules to cell surface CD8 (Ogg and McMichael, supra, 1998). The designation “m” is used to indicate that the class Ia molecule is a mutant; for example, HLA-A*0201m is generated from HLA-A*0201 by introducing an A245V substitution (see, for example, Bodinier et al., Nat. Med. 6:707-710, 2000). MHC tetramers containing mutated HLA molecules have a greatly diminished binding to the general population of CD8 cells, but retain peptide-specific binding, thus facilitating accurate discrimination of rare, specific T cells (less than 1% of CD8+; Altman et al., supra, 1996). For example, MHC tetramers composed of four HLA-A*0201 MHC class Ia molecules, each bound to a specific peptide and conjugated with phycoerythrin (PE), have been prepared (“i TAg™ MHC Tetramer”′; Immunomics/Beckman Coulter, Inc.). The HLA-A0201 allele is found in about 40% to 50% of the global population, and has been modified to minimize CD8 mediated binding (Bodinier et al., Nat. Med. 6:707-710, 2000, which is incorporated herein by reference). These complexes bind to a distinct set of T cell receptors (TCRs) on a subset of CD8+ T cells (McMichael and O'Callaghan, J Exp. Med. 187:1367-1371, 1998, which is incorporated herein by reference). The i TAg™ MHC Tetramer complexes, for example, recognizes human CD8+ T cells that are specific for the particular peptide and HLA molecule in the complex. Since specific binding does not depend on a functional pathway, the population identified by these tetramers includes all specific CD8+cells, regardless of functional status.

The monomers of an MHC tetramer or other polymer can be operatively linked together covalently or non-covalently, and directly through a physical association or chemical bond or indirectly through the use of a specific binding pair. As used herein, the term “operatively linked” or “operatively associated” means that a first molecule and at least a second molecule are joined together, covalently or non-covalently, such that each molecule substantially maintains its original or natural function. For example, where two MHC monomers, each of which can specifically bind a peptide antigen, are operatively linked to form an MHC dimer, each MHC monomer in the MHC dimer maintains its ability to specifically bind the peptide antigen. Any means can be used for operatively linking the monomers, provided it does not substantially reduce or inhibit the ability of an MHC polymer to present an antigenic peptide to a T cell. Generally, the MHC monomers are linked together through the heavy chain component of the monomers. Thus, the monomers can be linked, for example, through an interchain peptide bond formed between reactive side groups of the amino acids comprising the heavy chains, through interchain disulfide bonds formed between cysteine residues in the heavy chains, or through any other type of bond that can generally be formed between the chemical groups represented by the amino acid side chains.

A convenient means for operatively linking the monomers of an MHC polymer utilizes a specific binding pair. As used herein, the term “specific binding pair” refers to two molecules that can specifically interact with each other. The two molecules of a specific binding pair are referred to as “members of a specific binding pair” or as “binding partners.” A specific binding pair is selected such that the interaction is stable under conditions generally used to perform an immunoassay. Numerous specific binding pairs are well known in the art and include, for example, an antibody that specifically interacts with an epitope and the epitope, for example, an anti-FLAG antibody and a FLAG peptide (Hopp et al., BioTechnology 6:1204 (1988); U.S. Pat. No. 5,011,912); glutathione and glutathione S-transferase (GST); a divalent metal ion such as nickel ion or cobalt ion and a polyhistidine peptide; or the like.

Biotin and streptavidin have been used to prepare MHC tetramers, and biotin and avidin also can be used. These specific binding pairs provide the advantage that a single avidin or streptavidin molecule can bind four biotin moieties, thus providing a convenient means to prepare MHC tetramers. Biotin can be bound chemically to the lysine residues of an MHC heavy chain or can be bound using an enzymatic reaction, wherein the heavy chain is modified to contain a peptide signal sequence comprising a biotinylation site for the enzyme BirA (see Altman et al., supra, 1996; Ogg and McMichael, supra, 1998). Alternatively, biotin can be linked to the β2-microglobulin, which has fewer lysine residues than an MHC heavy chain, or can be linked to a mutant β2-mircroglobulin, which has been mutagenized to contain only a single accessible lysine residue.

Where a β2-microglobulin/β2m-AP complex is an MHC complex, for example, an MHC class I monomer or MHC class I tetramer, the complex can further contain a peptide antigen, which is specifically bound by the peptide binding cleft of the MHC class I molecule. Since MHC class I tetramers generally are used to detect a particular T cell, the peptide antigen is selected based on the specificity of the T cells to be detected. Peptide antigens that are bound by MHC molecules and presented to T cells are well known in the art and include, for example, a MART1 specific peptide, an HIVgag specific peptide, an HIVpol specific peptide, and the like (see Example 1; see, also, Lang and Bodinier, Transfusion 41:687-690, 2001; Pittet et al., Intl. Immunopharm. 1:12351247, 2001; U.S. Pat. No. 6,037,135; Intl. Publ. No. WO 94/20127; Intl. Publ. No. WO 97/34617).

The present invention provides immunoassays for detecting the presence of free β2-microglobulin in a sample containing, or suspected of containing, β2-microglobulin/β2m-AP complexes. The immunoassays of the invention can be “sandwich” assays, wherein a second antibody is used to detect specific binding of a first antibody and free β2-microglobulin, or can be competition assays, wherein binding of a competitor β2-microglobulin by the “first” antibody is indicative of the presence of free β2-microglobulin in a sample.

A first antibody useful in a method of the invention specifically binds free β2-microglobulin, but does not bind β2-microglobulin that is in a complex, for example, with an MHC class I molecule. Although no mechanism is proposed for such specificity, one possibility is that the antibody recognizes an epitope of β2-microglobulin that is on a face of the three dimensional β2-microglobulin molecule that associates with the β2m-AP. Monoclonal antibody C21.48A is an example of an antibody that specifically binds free β2-microglobulin, but does not bind β2-microglobulin that is in a complex with an MHC class I molecule β2m-AP (Liabeuf et al., J. Immunol. 127:1542-1548, 1981; Devaux et al., Res. Immunol. 141:357-372, 1990, each of which is incorporated herein by reference). As such, C21.48A or an antibody having substantially the same specific binding activity of C21.48A can be used as a first antibody in a method or kit of the invention, as can an antibody raised against the epitope to which C21.48A specifically binds or against an anti-idiotype antibody raised against the C21.48A antibody.

The term “antibody” is used broadly herein to include polyclonal and monoclonal antibodies, as well as antigen binding fragments of such antibodies. Depending on the particular method of the invention, antibodies having various specificities can be useful, including an antibody, or antigen binding fragment thereof, that specifically binds free β2-microglobulin, but does not bind β2-microglobulin when it is in a complex with a β2m-AP (also referred to generally herein as a “first antibody”); and an antibody that binds β2-microglobulin, regardless of whether the β2-microglobulin is in a free form or complexed form, including when the β2-microglobulin is specifically bound by a first antibody, such an antibody being useful as a second antibody in a sandwich-type immunoassay.

The term “specifically binds” or “specifically interacts,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for a β2-microglobulin epitope are included within the definition of an antibody. The term “specifically binds” or “specifically interacts” is used similarly herein to refer to the interaction of members of a specific binding pair, as well as to an interaction between β2-microglobulin and a β2m-AP such as an MHC class I heavy chain.

The term “antibody” as used herein includes naturally occurring antibodies as well as non-naturally occurring antibodies, including, for example, single chain antibodies, chimeric antibodies, bifunctional antibodies and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281, 1989). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)).

An antibody having a desired specificity can be obtained using well known methods. For example, an antibody having substantially the same specific binding activity of C21.48A can be prepared using methods as described by Liabeuf et al. (supra, 1981) or otherwise known in the art (Harlow and Lane, Antibodies: A laboratory manual (Cold Spring Harbor Laboratory Press 1988)). For example, an antibody that specifically binds free β2-microglobulin, but not to a β2-microglobulin/MHC class I molecule complex can be obtained using β2-microglobulin or a peptide portion thereof as an immunogen and removing antibodies that bind with a β2-microglobulin/MHC class I molecule complex. A peptide portion of a β2-microglobulin molecule that is present, for example, in a spatial region of β2-microglobulin that binds to a β2m-AP such as an MHC molecule can be identified using crystallographic data or protein modeling methods (see, for example, Shields et al., J. Immunol. 160:2297-2307, 1998; Pedersen et al., Eur. J. Immunol. 25:1609, 1995; Evans et al., Proc. Natl. Acad. Sci., USA 79:1994, 1995; Garboczi et al., Proc. Natl. Acad. Sci., USA 89:3429-3433, 1992; Fremont et al., Science 257:919, 1992, each of which is incorporated herein by reference).

Modeling systems can be based on structural information obtained, for example, by crystallographic analysis or nuclear magnetic resonance analysis, or on primary sequence information (see, for example, Dunbrack et al., “Meeting review: the Second meeting on the Critical Assessment of Techniques for Protein Structure Prediction (CASP2) (Asilomar, California, Dec. 13-16, 1996). Fold Des. 2(2):R27-42, 1997; Fischer and Eisenberg, Protein Sci. 5:947-55, 1996; U.S. Pat. No. 5,436,850; Havel, Prog. Biophys. Mol. Biol. 56:43-78, 1991; Lichtarge et al., J. Mol. Biol. 274:325-37, 1997; Matsumoto et al., J. Biol. Chem. 270:19524-31, 1995; Sali et al., J. Biol. Chem. 268:9023-34, 1993; Sali, Molec. Med. Today 1:270-7, 1995a; Sali, Curr. Opin. Biotechnol. 6:437-51, 1995b; Sali et al., Proteins 23: 318-26, 1995c; Sali, Nature Struct. Biol. 5:1029-1032, 1998; U.S. Pat. No. 5,933,819; U.S. Pat. No. 5,265,030, each of which is incorporated herein by reference).

The crystal structure coordinates of the interface region of a β2-microglobulin/MHC class Ia molecule complex can be used to identify peptide portions of β2-microglobulin that interact with the MHC molecule in the complex and, therefore, can be used to raise an antibody that specifically binds to free β2-microglobulin, but not to β2-microglobulin bound to the MHC class Ia molecule. The structure coordinates of the protein at the interface can also be used to computationally screen small molecule databases to identify mimics that may be useful for raising such an antibody. Such mimics can be identified by computer fitting kinetic data using standard equations (see, for example, Segel, Enzyme Kinetics (J. Wiley & Sons 1975), which is incorporated herein by reference).

Computer programs for carrying out the activities necessary to identify relevant structures using crystal structure information are well known. Examples of such programs include, Catalyst Databases™ program—an information retrieval program accessing chemical databases such as BioByte Master File, Derwent WDI and ACD; Catalyst/HYPO™ program—generates models of compounds and hypotheses to explain variations of activity with the structure of drug candidates; Ludi™ program—fits molecules into the active site of a protein by identifying and matching complementary polar and hydrophobic groups; and Leapfrog™ program—“grows” new ligands using a genetic algorithm with parameters under the control of the user.

The ability of a proposed peptide portion of β2-microglobulin to specifically bind an antibody such as C21.48A can be examined using any of several methods to screen molecules for their ability to specifically interact. This process may begin by visual inspection, for example, of a representation of a peptide portion of β2-microglobulin and the C21.48A mAb on a computer screen. Selected peptide portions of β2-microglobulin that potentially can be specifically bound the mAb then can be positioned in a variety of orientations, or docked, within an individual binding site of the mAb. Docking can be accomplished using software such as Quanta and Sybyl, followed by energy minimization and molecular dynamics with standard molecular mechanics forcefields, such as CHARMM and AMBER.

Specialized computer programs can be particularly useful for selecting peptide portions of β2-microglobulin useful for raising an antibody having the desired specificity. Such programs include, for example, GRID (Goodford, J. Med. Chem., 28:849-857, 1985; available from Oxford University, Oxford, UK); MCSS (Miranker and Karplus, Proteins: Structure. Function and Genetics 11:29-34, 1991, available from Molecular Simulations, Burlington Mass.); AUTODOCK (Goodsell and Olsen, Proteins: Structure. Function, and Genetics 8:195-202, 1990, available from Scripps Research Institute, La Jolla Calif.); DOCK (Kuntz, et al., J. Mol. Biol. 161:269-288, 1982, available from University of California, San Francisco Calif.), each of which is incorporated herein by reference.

Where a peptide portion of β2-microglobulin used as the immunogen is non-immunogenic, it can be made immunogenic by coupling the hapten to a carrier molecule such as bovine serum albumin (BSA) or keyhole limpet hemocyanin (KLH), or by expressing the peptide portion as a fusion protein. Various other carrier molecules and methods for coupling a hapten to a carrier molecule are well known in the art (see, for example, by Harlow and Lane, supra, 1988). Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1).

Monoclonal antibodies also can be obtained using methods that are well known and routine in the art (Kohler and Milstein, Nature 256:495, 1975; Coligan et al., supra, 1992, sections 2.5.1-2.6.7; Harlow and Lane, supra, 1988). For example, spleen cells from a mouse immunized with β2-microglobulin, or an epitopic fragment thereof, can be fused to an appropriate myeloma cell line such as SP/02 myeloma cells to produce hybridoma cells. Cloned hybridoma cell lines can be screened using, for example, labeled β2-microglobulin to identify clones that secrete monoclonal antibodies having the appropriate specificity, and hybridomas expressing antibodies having a desirable specificity and affinity can be isolated and utilized as a continuous source of the antibodies. Polyclonal antibodies similarly can be isolated, for example, from serum of an immunized animal. Such isolated antibodies can be further screened for the inability to specifically bind a β2-microglobulin/β2m-AP complex. Such antibodies, in addition to being useful for performing a method of the invention, also are useful, for example, for preparing standardized kits. A recombinant phage that expresses, for example, a single chain antibody also provides an antibody that can used for preparing standardized kits.

Monoclonal antibodies, for example, can be isolated and purified from hybridoma cultures by a variety of well established techniques, including, for example, affinity chromatography with Protein-A SEPHAROSE gel, size exclusion chromatography, and ion exchange chromatography (Barnes et al., in Meth. Mol. Biol. 10:79-104 (Humana Press 1992); Coligan et al., supra, 1992, see sections 2.7.1-2.7.12 and sections 2.9.1-2.9.3). Methods of in vitro and in vivo multiplication of monoclonal antibodies are well known. For example, multiplication in vitro can be carried out in suitable culture media such as Dulbecco's Modified Eagle Medium or RPMI 1640 medium, optionally replenished by a mammalian serum such as fetal calf serum or trace elements and growth sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, for example, syngeneic mice, to cause growth of antibody-producing tumors. Optionally, the animals can be primed with a hydrocarbon, for example, an oil such as pristane (tetramethylpentadecane) prior to injection. After one to three weeks, the desired monoclonal antibody is recovered from the body fluid of the animal.

An antigen binding fragment of an antibody that specifically binds free β2-microglobulin, but does not bind β2-microglobulin that is in a complex with a β2m-AP, also can be used in a method of the invention, as can an antibody derived from such an antibody, for example, a single chain antibody. An antigen binding fragment of an antibody can be prepared by proteolytic hydrolysis of a particular antibody such as C21.48A, or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647; Nisonhoff et al., Arch. Biochem. Biophys. 89:230. 1960; Porter, Biochem. J. 73:119, 1959; Edelman et al., Meth. Enzymol., 1:422 (Academic Press 1967); Coligan et al., supra, 1992, see sections 2.8.1-2.8.10 and 2.10.1-2.10.4).

Other methods of cleaving antibodies, such as separation of heavy chains to form monovalent light/heavy chain fragments, further cleavage of fragments, or other enzymatic, chemical, or genetic techniques can also be used, provided the fragments specifically bind to the antigen that is recognized by the intact antibody. For example, Fv fragments comprise an association of variable heavy (V_{H}) chains and variable light (V_{L}) chains, which can be a noncovalent association (Inbar et al., Proc. Natl. Acad. Sci., USA 69:2659, 1972). Alternatively, the variable chains can be linked by an intermolecular disulfide bond or cross-linked by chemicals such as glutaraldehyde (Sandhu, Crit. Rev. Biotechnol. 12:437, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are well known (see, for example, by Whitlow et al., Methods. A Companion to Methods in Enzymology 2:97, 1991; Bird et al., Science 242:423-426, 1988; Ladner et al., U.S. Pat. No. 4,946,778; Pack et al., Bio/Technology 11:1271-1277, 1993; Sandhu, supra, 1992).

Another example of an antigen binding fragment of an antibody is a peptide coding for a single complementarity determining region (CDR). CDR peptides can be obtained by constructing polynucleotides encoding the CDR of an antibody of interest. Such polynucleotides can be prepared, for example, using the polymerase chain reaction to synthesize a variable region encoded by RNA obtained from antibody-producing cells (see, for example, Larrick et al., Methods: A Companion to Methods in Enzymology 2:106, 1991, which is incorporated herein by reference).

Although not a necessity for in vitro uses, humanized monoclonal antibodies also can be used in a method or kit of the invention if desired. Humanized monoclonal antibodies can be produced, for example, by transferring nucleotide sequences encoding mouse complementarity determining regions from heavy and light variable chains of the mouse immunoglobulin into a human variable domain, and then substituting human residues in the framework regions of the murine counterparts. Methods for cloning murine immunoglobulin variable domains are known (see, for example, Orlandi et al., Proc. Natl. Acad. Sci., USA 86:3833, 1989), and for producing humanized monoclonal antibodies are well known (see, for example, Jones et al., Nature 21:522, 1986; Riechmann et al., Nature 332:323, 1988; Verhoeyen et al., Science 239:1534, 1988; Carter et al., Proc. Natl. Acad. Sci., USA 89:4285, 1992; Singer et al., J. Immunol. 150:2844, 1993; Sandhu, supra, 1992).

Antibodies useful in a method of the invention also can be derived from human antibody fragments, which can be isolated, for example, from a combinatorial immunoglobulin library (see, for example, Barbas et al., Methods: A Companion to Methods in Immunology 2:119, 1991; Winter et al., Ann. Rev. Immunol. 12:433, 1994). Cloning and expression vectors that are useful for producing a human immunoglobulin phage library are commercially available (Stratagene; La Jolla CA). In addition, the antibody can be derived from a human monoclonal antibody, which can be obtained from transgenic mice that have been “engineered” to produce specific human antibodies in response to antigenic challenge (see, for example, by Green et al., Nature Genet. 7:13, 1994; Lonberg et al., Nature 368:856, 1994; and Taylor et al., Int. Immunol. 6:579, 1994; see, also, Abgenix, Inc.; Fremont Calif.).

If desired, the antibody that binds free β2-microglobulin, but not β2-microglobulin when it is complexed with a β2m-AP, can be immobilized to a solid support. The solid support can be any material that is substantially insoluble under the conditions to which a method of the invention will be performed, i.e., under conditions in which immunoassays generally are performed. In addition, a material is selected as a solid support based on its stability to conditions under which an antibody is to be immobilized to the support. Thus, a solid support can be composed of glass, silicon, gelatin, agarose, a metal, or a synthetic material such as a plastic or other polymer, for example, polystyrene, polydextran, polypropylene, polyvinyl chloride, polyvinylidene fluoride, polyacrylamide, and the like.

Where the solid support has a hydrophobic surface, an antibody can be immobilized to the support simply by contacting the antibody and the surface such that the antibody is immobilized through a hydrophobic interaction with the surface, as is typical for solid phase immunoassays. A solid support also can be modified to contain reactive groups that facilitate binding of an antibody to the support, thereby immobilizing the antibody. Alternatively, or in addition, the antibody can be modified to facilitate immobilization to the support, for example, by modifying the antibody to contain a member of a specific binding pair, wherein the second member of the binding pair is a component of the support. For example, the antibody can be covalently bound, for example, to a magnetic iron oxide bead, which can be modified to contain reactive amine groups or carboxyl groups (Pierce Chemical Co.) or a member of a specific binding pair such as streptavidin (Dynal Biotech), thereby immobilizing the antibody and also providing a convenient means to isolate the antibody, as well as any specifically bound β2-microglobulin, from a mixture by contacting the mixture with a magnet (see, for example, Bodinier et al., Nat. Med. 6:707-710, 2000).

The means for immobilizing a first antibody to a solid support can include means for operatively linking MHC monomers to form an MHC tetramer or other polymer. A first antibody can be linked directly to the molecules that form the surface of the solid support, for example, by a peptide bond or a disulfide bond or the like formed between a reactive group of the molecules forming the surface of the solid support and a reactive group of the antibody, for example, an N-terminal amino group, C-terminal carboxyl group, or a reactive side chain of an amino acid residue of the antibody and a corresponding reactive group on the molecules comprising the solid support, provided that the immobilization does not substantially alter the specificity of the antibody to bind free β2-microglobulin, but not to a β2-microglobulin component of a complex. Alternatively, the molecules forming the surface of the solid support or the antibody can be modified to contain a linker moiety, which provides a means to link the antibody to the surface of the solid support.

A linker moiety can be a molecule that has a first reactive group, which allows it to bind to the surface of a solid support, and a second reactive group, which allows it to bind the first antibody such that the antibody can be immobilized to the solid support without substantially altering the antibody specificity (i.e., operatively linked). Thus, a linker moiety can be any agent, including a homo-bifunctional agent or hetero-bifunctional agent, that can react with a functional group present on a surface of the solid support and with a functional group present in the antibody to be immobilized thereto. Examples of bifunctional cross-linking agents include N-succinimidyl (4-iodacetyl) aminobenzoate, dimaleimide, dithio-bis-nitrobenzoic acid, N-succinimidyl-S-acetyl-thioacetate, N-succinimidyl-3-(2-pyridyidithiol propionate), succinimidyl 4-(N-maleimidomethyl) cyclohexane-1-carboxylate and 6-hydrazinonicotimide (see, also,, Wong “Chemistry of Protein Conjugation and Cross-Linking,” (CRC Press 1991); Hermanson, “Bioconjugate Techniques” (Academic Press 1995), each of which is incorporated herein by reference). A linker moiety also can be an amino acid, peptide or polypeptide that can be expressed with the antibody as a fusion protein, for example, a terminal cysteine residue or a peptide containing a terminal cysteine residue, which can provide a thiol group that can react with a thiol-reactive group on the surface of a solid support. In addition to providing a means to link the first antibody to a surface of a solid support, a linker moiety can function as a spacer molecule such that specificity of antibody is not affected due to steric constraints.

Where a first antibody is to immobilized to a solid support, it can be immobilized prior to, during, or after one or more of the binding reactions. Thus, the first antibody, i.e., the antibody this specifically binds free β2-microglobulin, but not β2-microglobulin complexed with a β2m-AP, can be immobilized prior to contacting it with the sample; during the time it is contacted with the sample, including, depending on the particular immunoassay method, during the time a competitor β2-microglobulin or a second antibody is contacted with the first antibody and sample; or it can be immobilized after the binding reaction is completed, or has reached a steady-state, and prior to detecting specific binding. When immobilization of the first antibody is performed during or after the contacting step, the means of immobilization is selected such that does not affect a specific binding interaction relevant to the immunoassay, for example, specific binding of the first antibody and free β2-microglobulin, or specific binding of the first antibody and competitor β2-microglobulin, or specific binding of a second antibody and a first antibody/β2-microglobulin complex.

A method of the invention is performed under any conditions typically used to perform an immunoassay, including a sandwich immunoassay or a competition immunoassay (see Example 2). As such, the reaction can be performed at a temperature of about 4° C. to 37° C., including, for example, at room temperature (about 18° C. to 23° C.), and for a period of time of about 30 minutes to 24 hours, for example, about 1 hour, or overnight (about 12 to 18 hours). The reaction also is performed generally in an aqueous solution, which can contain a buffer such that the pH of the reaction is maintained, if desired, in a relatively narrow range, for example, within about one pH unit of about pH 5, pH 7, or pH 9, and further can contain about a physiological concentration of sodium chloride or other suitable salt.

Detecting specific binding of the first antibody to free β2-microglobulin can be quantitative or qualitative, and can be performed in various ways. In one embodiment, a method of the invention is performed in a sandwich assay format, wherein binding of the first antibody, which specifically binds free β2-microglobulin, but does not substantially bind β2-microglobulin when it is complexed with a β2m-AP, is detected using a second antibody, which can specifically bind a complex formed by the specific binding of the first antibody to free β2-microglobulin. The second antibody also can, but need not, have the ability to specifically bind free β2-microglobulin, or free β2-microglobulin and β2-microglobulin that is complexed with a β2m-AP, provided the second antibody can specifically bind a complex of the first antibody and free β2-microglobulin. For example, the second antibody can be the monoclonal antibody B1G6, which specifically binds to free β2-microglobulin as well as to a β2-microglobulin/MHC class I molecule complex (Liabeuf et al., supra, 1981), or can be an antibody having substantially the same specific binding activity of B1G6.

In one aspect of a sandwich assay method of the invention, specific binding of the first antibody to free β2-microglobulin is detected by further contacting the sample and first antibody with a second antibody that specifically binds β2-microglobulin, including β2-microglobulin that is specifically bound to the first antibody, under conditions that allow specific binding of the second antibody; isolating the first antibody, including β2-microglobulin specifically bound to the first antibody and second antibody specifically bound thereto; and detecting second antibody associated with the isolated first antibody and β2-microglobulin. In another aspect, specific binding of a first antibody to free β2-microglobulin is detected by isolating the first antibody, including β2-microglobulin specifically bound thereto, from material not specifically bound to the first antibody; then contacting the isolated first antibody, including any β2-microglobulin specifically bound thereto, with the second antibody under conditions that allow specific binding of the second antibody to a complex comprising the first antibody and β2-microglobulin, and detecting second antibody associated with the isolated first antibody.

Isolating the first antibody can be performed using any method, and preferably is performed by immobilizing the first antibody, either directly or indirectly, to a solid support. For example, the first antibody can be immobilized by contacting it with a plastic surface such as the surface of the wells in a 96 well plate, wherein the first antibody interacts hydrophobically with the plastic surface, thereby immobilizing the antibody. The wells (or other surface formation) then can be washed to remove antibody that is not immobilized, and the wells can be further contacted with a blocking agent such as bovine serum albumin to reduce or inhibit non-specific binding of β2-microglobulin, for example, in a sample, with the surface (see Example 2).

It will be recognized that, where a second antibody specifically binds only a complex of the first antibody and β2-microglobulin, i.e., the second antibody does not specifically bind free β2-microglobulin or a β2-microglobulin/β2m-AP complex, isolating the first antibody/β2-microglobulin complex can be accomplished, for example, by immobilizing, the second antibody. When a method of the invention is performed using such a second antibody, the second antibody is not detectably labeled. Instead, the first antibody can be detectably labeled, or a third antibody (or an Fc receptor or other reagent that specifically binds the second antibody) can be used for the detecting step, wherein the third antibody specifically binds the first antibody and is detectably labeled. Such a second antibody also can be used to isolate a complex comprising a first antibody and a detectably labeled competitor β2-microglobulin, if desired.

In another embodiment, a method of the invention is performed in a competition assay format, wherein specific binding of competitor β2-microglobulin to a “first antibody,” which specifically binds free β2-microglobulin, but does not substantially bind β2-microglobulin when it is present as a β2-microglobulin/β2m-AP complex, is indicative of the presence or amount of free β2-microglobulin in a sample. A competition assay method of the invention is performed, for example, by contacting the sample, the first antibody, and a predetermined amount of competitor β2-microglobulin under conditions that allow specific binding of the antibody and β2-microglobulin; and detecting competitor β2-microglobulin specifically bound to the antibody, wherein the amount of competitor β2-microglobulin binding is indicative of the presence and, if desired, the amount, of free β2-microglobulin in the sample.

The competitor β2-microglobulin can be naturally-occurring β2-microglobulin that is isolated from cells or a biological fluid of an organism that normally produces β2-microglobulin, or can be recombinant β2-microglobulin that is expressed from a cloned encoding nucleic acid molecule. The β2-microglobulin, including naturally-occurring or recombinant β2-microglobulin, can be isolated from or expressed from a nucleic acid molecule isolated from any organism that expresses β2-microglobulin, particularly a vertebrate organism, including a mammal, for example, from human cells, murine cells, or the like. Cloned nucleic acid molecules encoding β2-microglobulin are well known and readily available to those in the art (see, for example, GenBank Accession Nos. XM_{—}007650, NM_{—}004048, and NM_{—}009735). An advantage of expressing a competitor β2-microglobulin as a recombinant polypeptide is that a peptide tag or other peptide detectable moiety, or ligand or substrate thereof, readily can be introduced into the competitor.

An anti-idiotype antibody also can be used as a “competitor β2-microglobulin” in a method or kit of the invention. The anti-idiotype antibody can be raised against the first antibody used in a method or kit of the invention, and can be selected based, for example, on having an affinity and kinetics of reactivity for binding to the first antibody that is substantially the same as that of free β2-microglobulin and the first antibody; or, where the kinetics or affinity of reactivity of the first antibody and anti-idiotype antibody are different from that of the first antibody and free β2-microglobulin, the difference is consistent and can be corrected for using routine methods such as adjusting the concentration of the reactants or using an algorithm to standardize the results. Similar considerations are made, for example, where the competitor β2-microglobulin is a detectably labeled polypeptide, since a detectable moiety, particularly a relatively large moiety such as an enzyme, can affect specific binding of the first antibody with a labeled competitor β2-microglobulin as compared to free (unlabeled) β2-microglobulin.

A second antibody or competitor β2-microglobulin generally, though not necessarily, contains a detectable label or other tag, which facilitates qualitative or quantitative detection of the free β2-microglobulin. The detectable label or tag can be any molecule generally used for such a purpose, for example, a fluorescent molecule, a radionuclide, a luminescent molecule, a chemiluminescent molecule, an enzyme, or a peptide such as a polyhistidine tag, a myc epitope, or a FLAG™ epitope. It should be recognized that, in many cases, a β2-microglobulin/β2m-AP complex such as an MHC tetramer in a sample that is being examined for the presence of free β2-microglobulin, also can be detectably labeled. As such, it can be desirable, depending on the particular format in which the method of the invention is performed, to select a detectable label for the second antibody (or competitor β2-microglobulin, where relevant) that is different from the label on the complex. For example, MHC tetramers comprising a fluorescent phycoerythrin label are commercially available (Immunomics). Thus, where a sample containing such a tetramer is being examined according to a method of the invention, the competitor β2-microglobulin or second antibody, for example, preferably is detectably labeled with a fluorescent molecule having an emission spectrum different from that of phycoerythrin, or is labeled with a moiety other than a fluorescent molecule, for example, with an enzyme.

Various detectable labels are known in the art and can be used for purposes of the present invention. Radionuclides such as tritium, carbon-14, phosphorous-32, iodine-125, iodine-131, and the like, are readily detectable using equipment that is generally available in research and clinical laboratories. In addition, methods for linking radionuclides to proteins such as an antibody or a β2-microglobulin polypeptide are well known. For example, iodine-125 or iodine-131 can be linked to an antibody using the chloramine-T procedure or lactoperoxidase procedure. A chromogenic molecule, which absorbs light in the visible or ultraviolet wavelength, also can be used, for example, a dye such as a quinoline dye, triarylmethane dye, phthalein, insect dye, azo dye, anthraquinoid dye, and the like. a fluorescent compound useful as a detectable label includes, for example, phycoerythrin, rhodamine, fluorescein, and umbelliferones, as well as fluorescent polypeptides such as a green fluorescent protein or a derivative or modified form thereof (see, for example, Langone et al., Meth. Enzymol. 74:3-105, 1981; U.S. Pat. Nos. 4,366,241; 3,996,345; 6,066,476).

An enzyme-catalyzed detection system can provide a particularly sensitive detection method. Enzyme labels are well known and include, for example, alkaline phosphatase, horseradish peroxidase, luciferase, β-galactosidase, glucose oxidase, lysozyme, malate dehydrogenase, and glucose-6-phosphate dehydrogenase (see, for example, U.S. Pat. Nos. 4,366,241; 4,740,468). Methods and reagents for linking an enzyme to a polypeptide such as an antibody also are well known and include, for example, glutaraldehyde, p-toluene diisocyanate, various carbodiimide reagents, p-benzoquinone, m-periodate, and N, N¹-o-phenylenedimaleimide. In addition, a fusion protein including, for example, β2-microglobulin and alkaline phosphatase can be prepared and expressed using recombinant DNA methods, thus providing a detectably labeled competitor β2-microglobulin.

The following examples are intended to illustrate but not limit the invention.

### EXAMPLE 1

### Stability of MHC Tetramers and MHC Monomers

This example provides an examination of MHC tetramer and MHC monomer stability using previously described methods.

Methods

Tetramer stability was examined biochemically by size exclusion chromatography (SEC) using an Superdex 75 HR 10×30 column. This method detects the free β2-microglobulin, which is an indicator of the dissociation from an MHC class Ia molecule. Areas under the peaks were integrated automatically using Millennium software. The quantity of β2-microglobulin was measured taking into account the area under the peaks corresponding to the elution time of the β2-microglobulin. Different quantities of purified β2-microglobulin were run, including solutions containing 40, 20, 10, 5, or 2.5 μg/ml (50 μl injection), as well as two internal standards, which were run at each time point.

Tetramers also were examined functionally using a flow cytometry method. The cell lines used to test the tetramers were mammalian cells transfected with a human TCR (VαVβ), which is specific for the HLA-A*0201/peptide combination. The stained cells were analyzed on an EpicsXL cytometer. Such cell lines were available only for the HIVpol and Mart1 tetramers. As such, the HLA-A*0201/HIVgag tetramer could not be tested by flow cytometry during this study. Tetramers were prepared in presence and absence of CD8, in order to evaluate the effect of the anti-CD8-FITC antibody on the stability of the tetramer.

SEC also was used to examine the dissociation of MHC monomers. Dissociation of the monomer was observed at t°>4° C. This dissociation process was temperature dependent, and peptide dependent (high affinity peptides are more stable as compared to low affinity peptides). Dissociation of the monomers was examined using a Superdex 200 HR 10×30 column, which allows detection of free β2-microglobulin and heavy chain (class Ia molecule) aggregates. Experiments were performed as for tetramers.

Results

A. Tetramers

1. Mutated HLA-A*0201/Mart1

All flow cytometry experiments were carried out with a human Jurkat T cell leukemia cell line. A TCR deficient Jurkat cell subclone, which is deficient for the endogenous Vβ8, was used to introduce the cloned α and β chains of the human TCR in a retroviral vector (MFG vector series; Dranoff, et al., Proc. Natl. Acad. Sci., USA 90:3539, 1993). The Jurkat cell line is CD3+, CD4+, CD8+, Vβ6.7+. The TCR recognizes the MelanA “wild type” peptide with very low affinity, whereas the decamer and the mutated peptide (26-35L, also called 27L) are well recognized.

Stability Results.

Tetramers with anti-CD8-FITC mAb as well as without anti-CD8-FITC mAb were prepared and studied. The effect of three different temperatures (4° C., 25° C., 37° C.) on the stability of the tetramers was tested. The 4° C. temperature represents the real time stability and 25° C. and 37° C. temperatures represent the stability under accelerated conditions. The tetramers without CD8 were studied only at 4° C. and the tetramers with CD8 (final product) were studied at 4° C., 25° C. and 37° C. The different lots of MHC tetramers (Immunotech/Beckman Coulter) used in this study are shown in Table I.

TABLE I
Lot number of the tetramers used in the study

| Tetramer | Lot Code |
|---|---|
| HLA-mA*0201/Mart1 | M00.002A without anti-CD8 |
| M00.002 (Lot 1) | M00.002B with anti-CD8 |
| HLA-mA*0201/Mart1 | M00.058A without anti-CD8 |
| M00.058 (Lot 2) | M00.058B with anti-CD8 |
| HLA-mA*0201/Mart1 | M00.059A without anti-CD8 |
| M00.059 (Lot 3) | M00.059B with anti-CD8 |

Generation of a dose-response curve of results obtained by flow cytometry after staining of the Jurkat cells at day 0 with the six different tetramers revealed only a small difference in the signal between tetramers with anti-CD8 mAb and without anti-CD8 mAb at Day 0. The percentage of the coefficient of variation was calculated and is shown in Table II. The statistical analysis of the results obtained at Day 0 for the different tetramers demonstrate that the coefficient of variation is low, suggesting strong reproducibility of the manufacturing process.

TABLE II
Ratio MFI Tetramer/MFI anti-CD3-PE

| μg/ml | M00.008A | M00.008B | M00.058A | M00.058B | M00.059A | M00.059B | Mean | Std | % CV |
|---|---|---|---|---|---|---|---|---|---|
| 40 | 1.28 | 1.15 | 1.27 | 1.06 | 1.22 | 1.00 | 1.163 | 0.114 | 9.85 |
| 20 | 1.15 | 1.11 | 1.17 | 1.05 | 1.14 | 1.00 | 1.103 | 0.065 | 5.94 |
| 10 | 1.06 | 0.99 | 1.04 | 1.00 | 1.05 | 0.96 | 1.016 | 0.039 | 3.86 |
| 5 | 0.93 | 0.89 | 0.95 | 0.90 | 0.90 | 0.87 | 0.906 | 0.028 | 3.17 |
| 2.5 | 0.82 | 0.79 | 0.81 | 0.80 | 0.78 | 0.77 | 0.795 | 0.018 | 2.35 |
| 1.25 | 0.69 | 0.69 | 0.69 | 0.68 | 0.65 | 0.67 | 0.678 | 0.016 | 2.36 |
| 0 | 0 | 0 | 0 | 0 | 0 | 0 | 0 | 0 | 0 |

After 180 days of incubation at 4° C., no major variation was found when staining the cells with the first lot of MHC tetramer, with or without CD8 This result indicates that the tetramer is stable at 4° C. and that the expression of the TCR, detected with the anti-CD3-PE, was also very stable (%CV=6 to 12%). When the results were plotted as the % of the control, all curves were parallel with a relatively small difference estimated at about 10%. These results indicate that the MHC tetramers, despite the presence or absence of CD8-FITC mAb, behave similarly at different concentrations. To determine % control, the Day 0 ratio represented the 100% control value; for all other time points (“Day X”), the % was compared to the % of the Day 0 value, calculated as follows: {day X (ratio mean fluorescence intensity, MFI, tetramer/MFI anti-CD3)×100}/(Day 0 MFI tetramer/MFI anti-CD-3).

A loss of about 20% of the signal was detected when MHC tetramers were incubated at 25° C. for 15 days, and a 25% loss after 180 days of incubation at 25° C. When plotted as the % of the control (see legend for the definition) as a function of the concentration of the tetramer, all of the curves were parallel, with a difference between day 0 and day 180 estimated at about 30%. These results indicate that the various MHC tetramers behave similarly at different concentrations of testing.

The effect of higher temperature on the stability of the tetramers was marked. MHC tetramers stored at 37° C. showed a drastic loss of signal obtained by flow cytometry after 7 days, and no signal was detected after 15 days. The standard deviation was extremely high when comparing the ratio obtained with three lots at day 7, suggesting a non-homogenous degradation pattern. However, the standard deviation was closer when the percentage of the control as a function of the concentration of the tetramer was compared. Interestingly, the loss of the signal correlated with the appearance of the free β2-microglobulin detected by size exclusion chromatography, as well as the increase of aggregates in the tetramer solution as measured by spectrometry. When the percentage of the control obtained with 1 μg/test of Day 0 to Day 15 of MHC tetramers incubated at 4° C., 25° C. or 37° C. was plotted against the level of free β2-microglobulin, the results from tetramers incubated at 4° C. and 25° C. were relatively grouped, suggesting a non-dissociation of the monomer within the tetramer complex. This result was confirmed upon analysis of staining obtained with the tetramers at 1 μg/test as a function of time; MHC tetramers, with or without CD8, stored at 4° C. were stable, while tetramers stored at 25° C. and 37° C. showed more or less loss of staining intensity depending on the temperature. No major differences were found between tetramers in the presence or absence of anti-CD8 antibody.

2. Mutated HLA-A*0201/HIVpol

The 80210 cell line, which is a rat basophil leukemia (RBL) cell line transfected with two hybrid constructs of human TCR alpha (Vα2.2) and beta (Vβ1) chains respectively, fused to the mouse TCR zeta chain (Engel et al., Science 256:1318, 1992), was used for these studies. This cell system allows expression of TCR without the context of the CD3 complex. Zeta chains form dimers expressed at the cell surface. The line can potentially express αα and ββ homodimers in addition to αβ heterodimers, however, their presence is difficult to demonstrate. There is neither CD3 nor CD8 expression on this cell line. The human TCR (Vα2.2/Vβ1) is specific for the HLA-A*0201/HIVpol combination. The 80210 cell line is adherent, and degranulates upon stimulation; for cytometry, a scatter change upon incubation with anti-TCR reagents was observed.

An anti-TCR Vβ1-PE (phycoerythrin) monoclonal antibody was used at saturation to examine the level of expression of the TCR on the cell line. As the tetramer recognizes the αβ component of the TCR that has the same stoichiometry as compared to TCR Vβ1 intensity, the monoclonal antibody anti-TCR Vβ1-PE serves as a calibrator of this assay. The stained cells were analyzed on an EpicsXL cytometer.

MHC tetramers with or without anti-CD8-FITC mAb were prepared, and the effect of three different temperatures (4° C., 25° C., 37° C.) in the stability of the tetramers with or without CD8 was tested. The 4° C. represent the real time stability and 25 and 37° C. represent the stability under accelerated conditions. Table III summarizes the different lots of the tetramer HLA-A*0201/HIVpol used during the study.

TABLE III
Lot number of the tetramers used in the study

| Tetramer | Lot No. |
|---|---|
| HLA-mA*0201/HIVpol | M00.007A with anti-CD8 |
| M00.007 (Lot 1) | M00.007B without anti-CD8 |
| HLA-mA*0201/HIVpol | M00.028A without anti-CD8 |
| M00.028 (Lot 2) | M00.028B with anti-CD8 |
| HLA-mA*0201/HIVpol | M00.029A without anti-CD8 |
| M00.029 (Lot 3) | M00.029B with anti-CD8 |

A dose-response curve obtained by flow cytometry after staining of the RBL 80210 cells at day 0 with the six different tetramers revealed a very close correspondence between MHC tetramers with anti-CD8 and MHC tetramers without anti-CD8 at day 0. A slight difference was found with the first lot of tetramer without anti-CD8 Ab. Table IV shows the percentage of the coefficient of variation for all lots of tetramer HIV/pol analyzed at day 0 and at different concentrations. Except at the last dilution (1.25 μg/ml %CV 10%), the % of the CV of other concentrations ranged between 3 and 5%, suggesting strong reproducibility of the manufacturing process.

TABLE IV
Ratio MFI Tetramer/MFI anti-Vβ1-PE

| μg/ml | M00.007A | M00.007B | M00.028A | M00.028B | M00.029A | M00.029B | Moyenne | ecartype | % CV |
|---|---|---|---|---|---|---|---|---|---|
| 40 | 0.63 | 0.71 | 0.73 | 0.7 | 0.72 | 0.7 | 0.698 | 0.035 | 5.076 |
| 20 | 0.56 | 0.64 | 0.65 | 0.65 | 0.66 | 0.63 | 0.631 | 0.036 | 5.787 |
| 10 | 0.51 | 0.55 | 0.52 | 0.53 | 0.55 | 0.55 | 0.535 | 0.017 | 3.290 |
| 5 | 0.38 | 0.42 | 0.42 | 0.43 | 0.43 | 0.42 | 0.416 | 0.018 | 4.468 |
| 2.5 | 0.28 | 0.32 | 0.31 | 0.29 | 0.32 | 0.3 | 0.303 | 0.016 | 5.383 |
| 1.25 | 0.19 | 0.21 | 0.16 | 0.2 | 0.18 | 0.21 | 0.191 | 0.019 | 10.12 |
| 0 | 0 | 0 | 0 | 0 | 0 | 0 | 0 | 0 | 0 |

Results were somewhat different when comparing the ratios obtained at different times between the tetramers in presence or absence of the anti-CD8 antibody and stored at 4° C. However, when the % control as a function of the concentration at different times was compared, the tetramers stored at 4° C. with the anti-CD8 antibody were relatively more stable than the tetramer without the anti-CD8 antibody. A comparison with the % control at 4° C. with 1 μg/test as a function of time showed no major differences. After 6 months, there was a loss of≦10% on these tetramers.

A variation of the signal obtained at 25° C. also was observed, and a very strong effect on the tetramers stored at 37° C., both in presence and absence of anti-CD8 antibody, was observed. There was a loss of 50% of the signal after 5 months (150 days) at 25° C. for the higher concentrations and 70% for the lower concentrations, for both types of tetramers. The tetramers incubated at 37° C. showed a drastic loss of the signal obtained by flow cytometry after 7 days, and no more signal was detected after 15 days. Like the MHC tetramer Mart1, the standard deviation was extremely high when comparing the ratio obtained with the three lots at day 7, suggesting a non-homogenous degradation pattern.

The results were confirmed by the measurement of the free β2-microglobulin by gel filtration chromatography and the measurement of aggregate formation. The quantity of the free β2-microglobulin of tetramers stored at 4° C. remained constant, and increased in MHC tetramers incubated at 25° C. and 37° C., either with or without CD8. The loss of the signal by flow cytometry correlated with the level of the free β2-microglobulin detected by size exclusion chromatography. The analysis of cells stained with 1 μg/test of tetramer as a function of time confirmed the previous observation. Tetramers incubated at 4° C. were stable, while tetramers incubated at 25° C. and 37° C. lost function with time. However, there were no major differences between the tetramers containing anti-CD8 compared to the tetramers without CD8.

Open Vial Study of HLA-A*0201/HIVpol

One lot of MHC tetramer HLA-A*0201/HIVpol (Lot M00-007) containing the anti-CD8-FITC antibody was analyzed in an open vial study, as above. A variation of 10±5.6% was observed when compared to either the ratio of the MFI tetramer/MFI Vβ1 mAb or the % control in function of the tetramer concentration with the results obtained at day 0. The tetrarner worked well, with a loss of 10% of the staining intensity after 6 months. These results demonstrate that the MHC tetramer remained stable after weekly opening of the vial over a period of 3 months and showed the identical staining intensity after opening then from the closed vial.

3. Mutated HLA-A*0201/HIVgag

Lots of tetramer examined in this study are shown in Table V. This tetramer was studied only by biochemical techniques because no specific cell line was available. Similar to the other two tetramers, size exclusion chromatography revealed that the tetramer dissociated very much faster at 37° C. The level of the free β2-microglobulin tended to plateau; however tetramers in presence or absence of the anti-CD8 FITC mAb were very stable at 4° C. The dissociation of the tetramer correlated with the appearance of the aggregates and the decrease of the PE signal. No aggregates were observed in MHC tetramer either with or without CD8 and incubated at 4° C.

TABLE V
Lot number of the tetramers used in the study

| Tetramer | Lot No. |
|---|---|
| HLA-mA*0201/HIVgag | M00.001A without anti-CD8 |
| M00.001 [Lot 1] | M00.001B with anti-CD8 |
| HLA-mA*0201/HIVgag | M00.053A without anti-CD8 |
| M00.053 [Lot 2] | M00.053B with anti-CD8 |
| HLA-mA*0201/HIVgag | M00.054A without anti-CD8 |
| M00.054 [Lot 3] | M00.054B with anti-CD8 |

B. Monomers

The MHC monomer is the essential subunit to generate MHC tetramers. The monomer is composed of 1) an MHC class Ia heavy chain containing, at the carboxyl terminus, a specific sequence recognized by the enzyme BirA, which introduces a biotin moiety on a specific lysine, 2) a β2-microglobulin light chain, and 3) a specific peptide. The lack either of the peptide or the β2-microglobulin induces the dissociation and the final aggregation of the heavy chain.

Two different degradation phenomena have been identified for the monomer—dissociation of the monomer and debiotinylation of the heavy chain. These two aspects were studied using SDS-PAGE and SEC. Monomers were prepared and their stability was studied (Lot numbers used for this study are shown in Table VI). The effect of three different temperatures (−80° C., 4° C. and 25° C.) in the stability of the monomers was tested. The temperature −80° C. represents the real temperature of storage and 4° C. and 25° C. represent the stability under accelerated conditions.

TABLE VI
Lot number of the monomers used in the study

| Monomer Lot | Specificity |
|---|---|
| Lot 1 M00-008 | HLA-A*0201/Mart 1 |
| Lot 2 M00-043 | HLA-A*0201/Mart 1 |
| Lot 3 M00-044 | HLA-A*0201/Mart 1 |
| Lot 1 M00-007 | HLA-A*0201/HIVpol |
| Lot 2 M00-017 | HLA-A*0201/HIVpol |
| Lot 3 M00-019 | HLA-A*0201/HIVpol |
| Lot 1 M00-009 | HLA-A*0201/HIVgag |
| Lot 2 M00-030 | HLA-A*0201/HIVgag |
| Lot 3 M00-039 | HLA-A*0201/HIVgag |

The measurement of free β2-microglobulin by size exclusion chromatography provides an indication of the dissociation of the monomer. Results revealed that monomers stored at −80° C., 4° C. and 25° C. were stable. Some variation was observed in the sample analyzed after 15 days, however, the standard deviation was lower in most of the cases when the three different lots of monomer were analyzed at the same time. The comparison of the free β2-microglobulin levels in the monomers and the tetramer correlated well. However, in the samples stored at 4° C., irregularities appeared irregularities in the curves that may reflect the inaccuracy of the technique and errors during the manipulation (calculated as ±23% accurate). The level of free β2-microglobulin was equivalent for the four monomers.

Conclusions

Several conclusions were drawn from the study of tetramer and monomer stability for HLA-A*0201/HIVpol, HLA-A*0201/HIVgag and HLA-A*0201/Mart1.

Tetramers

1. All three MHC tetramers were stable at 4° C. after 6 months.

2. There was no major difference between tetramers with CD8 and tetramers without CD8 for the HLA-mA*0201/Mart1 tetramer. However, the HLA-mA*0201/HIVpol tetramer was relatively less stable without CD8.

3. MHC tetramers stored at 25° C. showed slow dissociation. The HLA-A*0201/HIVpol tetramer was less stable at accelerated temperatures than the HLA-A*0201/HIVgag and HLA-A*0201/Mart1 tetramers.

4. Tetramers stored at 37° C. showed very fast degradation and, after 7 days, were totally degraded.

5. Despite some variability, it was clear that the behavior of the 3 tetramers was highly comparable over the period of time studied at 4° C. However, the HLA-A*0201/HIV pol tetramer was slightly less stable at 25° C. compared to the HLA-A*0201/Mart1 and HLA-A*0201/HIVgag tetramers.

6. Based on the data obtained for all tetramers with and without CD8, the first lot of HLA-A*0201/HIVpol tetramers was validated for the purpose of demonstration of 6 months real time stability for the final product.

7. The accelerated data on all 3 lots were closely correlated for each specificity.

8. The results obtained with the different methodologies correlated well; high levels of free β2-microglobulin were found in tetramers in which the MFI diminished after incubation at 37° C. This observation is relevant because it allows a prediction of stability for tetramers in which a specific cell line was not available.

9. The measurement of PE aggregate formation also correlated also with the decrease of the signal in flow cytometry.

The results obtained with the open vial study demonstrated that this procedure did not induce a major modification in the capability of the tetramer to stain cells.

Monomers.

Monomers were stable at −80° C. as well as at 4° C. and 25° C., as shown by the gel filtration chromatography experiments as well as by the SDS-PAGE results. No debiotinylation of the monomer was detected in samples stored either at −80° C., 4° C. or 25° C., suggesting that the avidin gel purification removed a potential protease that previously was found to be involved in the degradation of the monomer.

### EXAMPLE 2

### Immunoassay Development and Characterization

The measurement of the free β2-microglobulin is an indicator of the dissociation of the monomer and the tetramer. During the development of HLA-A*0201/HIVgag, HLA-A*0201/HIVpol and HLA-A*0201/Mart1 complexes, free β2-microglobulin was established as the best correlate with integrity of the final as well as intermediate product. Currently, free β2-microglobulin is measured by gel filtration chromatography. However, the use of gel filtration chromatography (size exclusion chromatography; SEC) during stability studies, where large numbers of samples are processed at the same time, consumes long instrument times, which leads to high equipment costs, does not allow for doublet or triple testing due to time, is not very accurate (CV 28%) or very sensitive, and low level of monomer deterioration is not detected precisely. In addition, SEC requires a relatively large amount of material. The immunoassays disclosed herein provide significant advantages over the use of SEC to measure free β2-microglobulin, for example, in samples containing MHC monomers and MHC tetramers.

β2-microglobulin dissociation of the MHC monomer is observed at a temperature greater than 4° C., is temperature dependent, and is peptide dependent (high affinity peptides were more stable as compared to low affinity peptides). Dissociation of the MHC monomers previously has been studied by SEC, which allows detection of free β2-microglobulin as well as the aggregation of the MHC class Ia heavy chains (see Example 1). As discussed above, however, this technique presents several disadvantages. For example, the number of samples analyzed usually takes 24 to 48 hours, which does not allow for analysis of doublets. In addition, the variability of SEC is rather high—the precision of the calculated values was shown to 23% during validation. For these reasons, an easier, faster, and more accurate method was developed.

Immunoassay methods, including enzyme immunoassays (EIAs), were examined. Two principle types of immunoassays were examined, a sandwich assay and a competition assay. The “classic” sandwich method employs two different monoclonal antibodies raised against two different epitopes. This method is often very sensitive and is reliable. In comparison, the competition assay uses only one monoclonal antibody, and the antigen, which can be labeled with a radioisotope or coupled to an enzyme or other detectable label, is used as a tracer. This method is reliable, but is less sensitive than the sandwich immunoassay.

In order to be able to measure free β2-microglobulin without interference from β2-microglobulin complexed in an intact MHC monomer or MHC tetramer, an antibody is used that recognizes an epitope masked by the association of β2-microglobulin to the heavy chain. The C21.48A monoclonal antibody (mAb; Liabeuf et al., supra, 1981) is an example of such an mAb. Liabeuf et al. (supra, 1981) showed that C21.48A, which is a mouse IgG2b immunoglobulin, bound to free β2-microglobulin but, in contrast to several other mAbs, including B1G6, did not bind to β2-microglobulin that was associated with a cell surface. In addition, Devaux et al. (supra, 1990) showed that C21.48A, in contrast to several other mAbs, including B1G6, failed to interfere with the HIV 1 replicative cycle in the MT4 T leukemic cell line, whereas C21.48A mAb was devoid of a functional effect. These two different studies indicate that the C21.48A antibody is specific for a β2-microglobulin epitope involved in binding to the HLA class I heavy chain molecule.

Hybridoma cell lines expressing the B1.G6 antibody and the C21.48A antibody have been deposited Mar. 5, 2002 according to the terms of the Budapest Treaty with the Collection Nationale De Cultures De Microorganismes (CNDC) at the Institut Pasteur, 25/28 rue de Dr Roux, 75724 PARIS Cedex 15, which is a recognized depository, for a term of at least thirty years and at least five years after the most recent request for the furnishing of the deposit was received by the depository, and under conditions that assure access to the deposit during the pendency of the patent application as determined by the Commissioner, and upon request during the term of the patent. For the B1.G6 antibody, hybridoma clone B1.G6.31.29.1 was deposited as register number CNCM I-2813. For the C21.48A antibody, hybridoma clone C21.48A1.1 was deposited as register number CNCM I-2814. It will be recognized that the availability of the deposited clones provides a standard for the comparison of other antibodies, including those made using methods as disclosed herein or otherwise known in the art, to identify those having substantially the same specificity as the antibodies produced by the deposited hybridoma cell lines.

During feasibility studies, an EIA was used to quantitate the biotinylated monomers using the B1G6 mAb. Streptavidin-peroxidase was used to reveal the biotinylated product. An immunoassay for dosing the β2-microglobulin using the B1G6 mAb is commercially available (Immunotech/Beckman Coulter). The B1G6 mAb recognizes an epitope located outside the interface of interaction between the heavy chain and the β2microglobulin (Liabeuf, et al., supra, 1981. The C21.48A and B1G6 mAbs were used to develop an EIA for measuring free β2-microglobulin.

Material and Methods

Reagents and Antibodies

Human recombinant biotinylated-HLA-A*0201 monomers and human recombinant β2-microglobulin (rβ2m) were obtained from the manufacturing department of Immunomics (Marseille, France). Natural human β2-microglobulin purified from urine; purified B1G6 mAb (IgG2a; Lot# F1317-2); ammonium sulfate precipitate of ascites fluid of C21.48A mAb; streptavidin conjugated peroxidase, and peroxidase-conjugated anti-β2-microglobulin monoclonal antibody B1G6 were obtained from Immunotech.

Biotinylation of the Recombinant β2microglobulin

Human rβ2m was biotinylated with biotin-ε-amino-caproic acid-N-hydroxysuccinimide ester (Roche Diagnostic, Switzerland). Briefly, 1 mg of protein at 1 mg/ml in 50 mM borate, 0.15M NaCl (pH 8.8) was incubated with 5.6 μl of biotin-ε-amino-caproic acid-N-hydroxysuccinimide ester at 10 mg/ml. The reaction mixture was incubated for 20 min at 20° C. and the reaction stopped with 100 μl of 1 M NH₄Cl. Proteins and low molecular weight reactants were separated by dialysis in PBS for 16 hr at 4° C., aliquoted and frozen.

C21.48A mAb Purification

C21.48A mAb was purified by affinity chromatography using Protein A. Purity was controlled under reducing and non-reducing conditions with Nu-PAGE gels following the instructions provided by the manufacturer.

Recognition of the β2-microglobulin by the anti-β2microglobulin mAbs

96-well microtiter plates were coated with 100 μl of the human rβ2m at 5 μg/ml in PBS and blocked with 3% BSA in PBS. The assay procedure was as follows: 100 μl/well of several concentrations of anti-β2-microglobulin antibodies or control antibodies were incubated for 1 hr at room temperature (RT) on an orbital shaker. The wells were rinsed three times with an automatic washer (SLT; Salzburg, Austria) with 300 μl of a 9 g/l NaCl solution containing 0.05% Tween 80, and 100 μl/well of 1/5000 peroxidase-conjugated goat anti-mouse antibody were added. The plates were incubated for 30 min at room temperature on an orbital shaker, washed three times, and TMB peroxidase substrate was added. The color reaction was allowed to develop in the dark for 5 min with agitation. The reaction was then stopped by addition of 50 μl/well of 2N H₂SO₄ and the absorbance was measured at 450 nm with a microplate reader (Molecular Device, UK). The absorbance of the substrate was subtracted from all values. All determinations were performed in duplicate.

Binding of Biotinylated Monomers to anti-β2microglobulin Antibodies

96 well microtiter plates were coated with 100 μl of the anti-β2-microglobulin antibodies clone B1G6 or C21.48A at 5 μg/ml in PBS and blocked with dried buffer. The EIA was performed, using a solid phase coated with anti-β2-microglobulin antibodies (B1G6 or C21.48A). 100 μl/well of biotinylated monomer was incubated for 1 hr at RT on an orbital shaker. The wells were rinsed three times with an automatic washer with 300 μl of a 9 g/l NaCl solution containing 0.05% Tween 80, and 100 μl/well of streptavidin-peroxidase solution were added. The plates were incubated for 30 min at RT on an orbital shaker, washed three times and TMB peroxidase substrate was added. The color reaction was allowed to develop in the dark for 5 min with agitation. The reaction was stopped by addition of 50 μl/well of 2N H₂S0₄ and the absorbance was measured at 450 rum with a microplate reader (Molecular Device, UK). The absorbance of the substrate was subtracted from all values. All determinations were performed in duplicate.

Immunoassay Procedure

A. Sandwich EIA

96 well microtiter plates were coated with 100 μl of the anti-β2-microglobulin antibody C2148A at 5 μg/ml in PBS and blocked with dried buffer. 100 μl/well of different concentrations of human rβ2m were incubated for 1 hr at RT on an orbital shaker. The wells were rinsed three times with an automatic washer with 300 μl of a 9 g/l NaCl solution containing 0.05% Tween 80, and 100 μl/well of peroxidase conjugated B1G6 mAb solution were added. The plates were incubated for 1 hr at RT on an orbital shaker, washed three times, and TMB peroxidase substrate was added. The color reaction was allowed to develop in the dark for 5 min with agitation. The reaction was then stopped by addition of 50 μl/well of 2N H₂S0₄ and the absorbance was measured at 450 nm with a microplate reader. The absorbance of the substrate was subtracted from all values. All determinations ware performed in duplicate.

B. Competition EIA

96 well microtiter plates were coated with 100 μl of the anti-2-microglobulin antibody C21.48A at 5 μg/ml in PBS and blocked with dried buffer. 10 μl/well of different concentrations of human rβ2m or samples, and 200 μl/well of alkaline phosphatase-conjugated β2microglobulin solution were added. The plates were incubated for 90 min at RT on an orbital shaker. The wells were rinsed three times with an automatic washer with 300 μl of a 9 g/l NaCl solution containing 0.05% Tween 80, and pNPP substrate was added. The color reaction was allowed to develop in the dark for 30 min with agitation. The reaction was stopped by addition of 50 βl/well of 1 M NaOH and the absorbance was measured at 405 nm with a microplate reader. The absorbance of the substrate was subtracted from all values. All determinations were performed in duplicate.

Results

Recognition of the β2-microglobulin by B1G6 and C21.48A mAbs

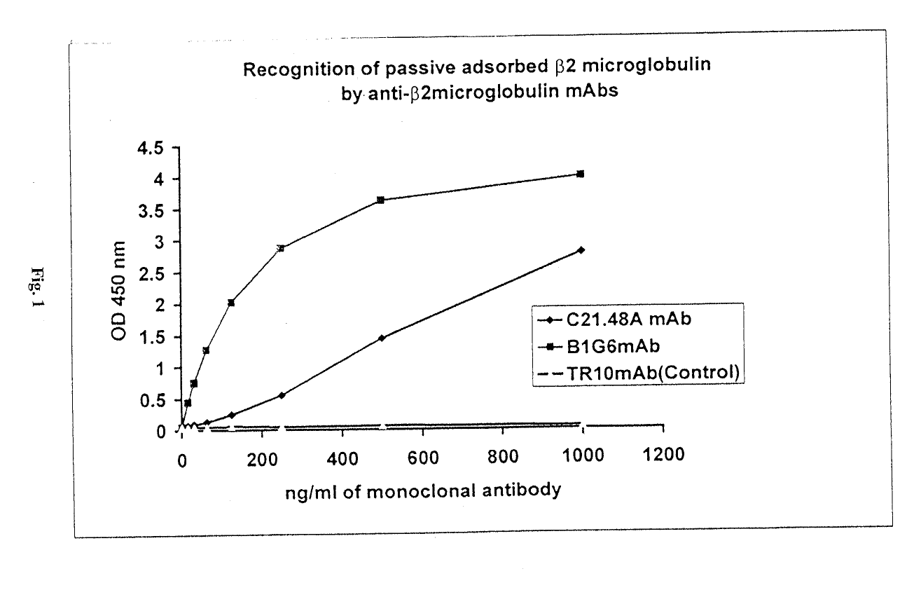

The ability of the anti-β2-microglobulin monoclonal antibody B1G6, recognizes an epitope located outside the interface of interaction between the heavy chain and the β2-microglobulin, and the anti-β2-microglobulin monoclonal antibody C21.48A, which recognizes an epitope involved in binding to the HLA class I heavy chain molecule, to recognize β2-microglobulin coated on a solid phase was examined, and compared to binding by an irrelevant antibody (TR10mAb). A dose-response curve was observed with the two anti-β2-microglobulin antibodies, whereas no signal was obtained in the wells incubated with the irrelevant antibody (FIG. 1).

The difference in the signal obtained with B1G6 and C21.48A mAbs likely was due to an alteration of a fraction of the β2-microglobulin by the solid phase; in this respect, it is well known that the immobilization of the proteins by passive adsorption on a plastic surface, as is the case of the present ELISA plates, is random, and that the fixation is generally due to hydrophobic interactions between the protein and the solid phase. As such, passive adsorption can result in the loss or alterations of antigenic epitopes or in steric hindrance. Taking into account that the interface interaction between the heavy chain and the β2-microglobulin, as revealed by X-ray crystallography, is basically governed by hydrophobic interactions, the present results are not discrepant.

Reactions Condition

Based on the characteristics of the mAbs used in this study, the position of each antibody in the ELISA scheme can be affect the assay. In one scenario, the B1G6 mAb is coated on the solid phase and C21.48A is the tracer. In this scheme, the B1G6 mAb can capture the β2-microglobulin associated to the heavy chain; the C21.48A mAb is unable to bind to the β2-microglobulin. Furthermore, when a fraction of the monomer is dissociated, the free β2-microglobulin can be detected; however, an excess of native monomer can saturate the binding sites of the B1G6 mAb and, therefore, the quantity of free β2-microglobulin in the sample can be underestimated.

In an alternate scenario, C21.48A mAb is coated on the solid phase and B1G6 is used as tracer. In this scheme, only the free β2-microglobulin is detected; the native monomer does not interfere in the measure of the free β2-microglobulin. However, in this scenario; the ELISA should be performed in two steps to avoid the loss of the B1G6 mAb captured by the native monomer. A first incubation step should be performed only in presence of the sample, and only after washing and the elimination of the excess of native monomer, the second anti-β2-microglobulin antibody B1G6 should be added during a second incubation step.

Binding of the Biotinylated Monomer to anti β2-microglobulin mAbs

From the different aspects described above and taking into account the characteristics of these antibodies, the ability of the anti-β2-microglobulin antibodies adsorbed onto a solid phase to recognize different biotinylated-monomers in solution was examined, as was the ability of a biotin tag on the C-terminus of the heavy chain to bind the streptavidin conjugated to the peroxidase. Several dilutions of 5 different purified biotinylated-monomers were examined (Table VII); the dilutions were incubated on a microtiter plate coated with the anti-β2-microglobulin antibodies, or an irrelevant antibody (an anti-IL-4R mAb).

TABLE VII
Biotinylated monomers

| Monomer Lot # | Specificity | Concentration | Date |
|---|---|---|---|
| M00-015 | HLA-mA*0201/HIVgag | 0.48 mg/ml | 26-27/06/00 |
| M00-100 | HLA-mA*0201/HIVgag | 0.52 mg/ml | 03-08/11/00 |
| M00-101 | HLA-mA*0201/HIVgag | 0.51 mg/ml | 03/08/11/00 |
| M00-110 | HLA-mA*0201/Mart1 | 0.48 mg/ml | 14-15/11/00 |
| M00-111 | HLA-mA*0201/Bmif1 | 0.49 mg/ml | 14-15/11/00 |

As expected, a dose-response curve was obtained, and reached a plateau at 10 ng/ml with the plates coated with the anti-β2-microglobulin B1G6 antibody; no signal was obtained with the C21.48A or anti-IL-4R mAbs. To demonstrate that the absence of signal in plates coated with C21.48A mAb was due to the absence of binding of the biotinylated monomer and not a problem of coating the plates, a biotinylated ,β2-microglobulin with a biotin/β2-microglobulin (1:1 ratio) was prepared. Dilutions of this biotinylated β2-microglobulin were prepared and incubated on a microtiter plate coated with the three different antibodies. A significant signal was detected in plates coated with the specific antibodies, whereas no signal was detected in plates coated with the irrelevant antibody. This result confirms that the absence of the signal in plates coated with C21.48A mAb and incubated with the biotinylated monomers was due to the absence of interaction between the biotinylated monomer and the C21.48A mAb, and was not a problem with the plates.

These results demonstrate that the biotinylated monomer captured by the anti-β2-microglobulin antibody B1G6 binds the streptavidin-peroxidase despite the capture of the monomer by the antibody. No steric hindrance was observed with this antibody. In addition, this result demonstrates that the anti-β2microglobulin antibody C21.48A can be used to measure the free β2-microglobulin and that the native monomer will not have an influence on the mAb.

Sandwich ELISA to Measure Free β2-microglobulin

A two step immunometric type assay was developed to measure the free β2-microglobulin, wherein a solid phase was coated with the C21.48A mAb and the B1G6 mAb antibody conjugated to peroxidase was used as a tracer. The assay was performed as described in Table VIII.

TABLE VIII
Summary of Sandwich Assay Procedure

| Step 1 | Step 2 | Step 3 | Step 4 | Step 5 | Step 6 |
|---|---|---|---|---|---|
| To well coated | Aspirate | Dispense | Aspirate | Dispense | Add 50 μl of |
| with | Rinse 3 | 100 μl/well of | Rinse 3 | 100 μl/well of | stop solution |
| monoclonal | times with | Peroxidase | times with | TMB substrate | H₂SO₄2N |
| antibody | 300 μl of | Conjugated | 300 μl of | Incubate 10 | Read at 450 nm |
| C21.48A add | wash | B1G6 | wash | min with | |
| 100 μl of | solution | monoclonal | solution | shaking in the | |
| standard or | | antibody at | | dark | |
| sample | | 10 ng/ml | | At 18-25° C. | |
| Incubate 60 | | Incubate 60 min | | | |
| min | | With shaking At | | | |
| With shaking | | 18-25° C. | | | |
| At 18-25° C. | | | | | |

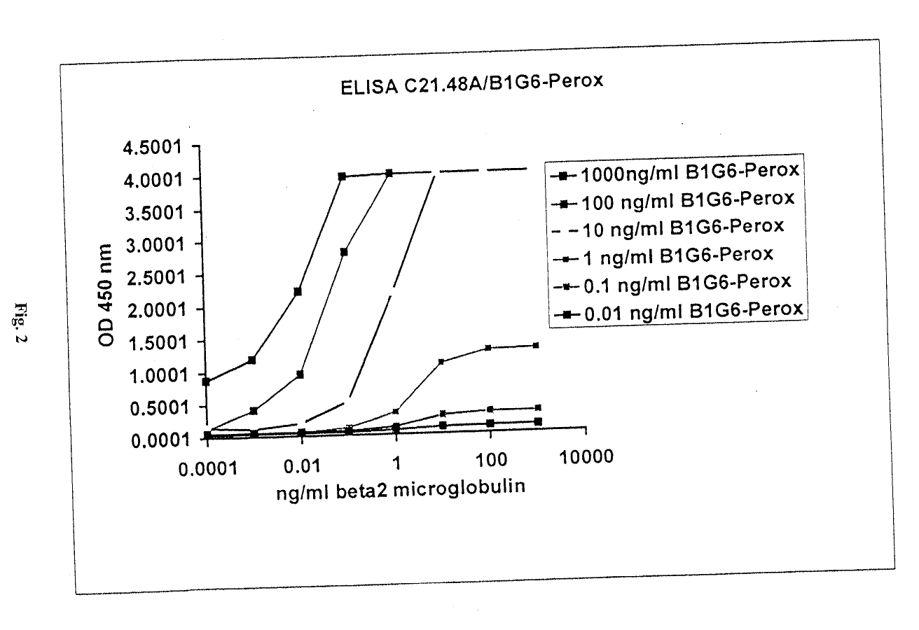

Serial dilutions of β2-microglobulin and serial dilutions of B1G6-peroxidase were tested in the C21.48A/B1G6-peroxidase assay, to determine the best concentration of the B1G6-peroxidase and the dynamic range of the standard curve. High background was observed at 1 μg/ml B1G6-Peroxidase (FIG. 2). At 100 ng/ml of B1G6-peroxidase, the signal was significantly greater than background when the β2-microglobulin concentration was greater than 1 pg/ml; at 10 ng/ml of B1G6-peroxidase, the signal was significantly greater than background when the β2-microglobulin concentration was greater than 15 pg/ml (FIG. 2). The signal was lower when B1G6-peroxidase was used at 1, 0.1 and 0.01 ng/ml. These results indicate that, unless an extremely sensitive assay is required, the sensitivity obtained with 100 ng/ml of B1G6-peroxidase is too high.

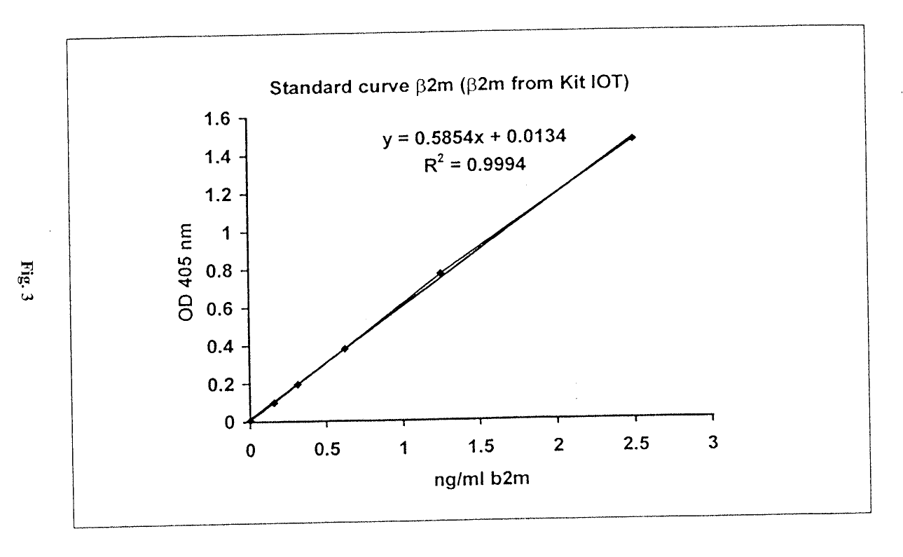

Based on the above results, further experiments were performed using a final concentration of 1 ng/ml of B1G6-peroxidase. Under these conditions, a linear dose response curve was obtained up to 2.5 ng/ml of β2-microglobulin; the minimal detectable amount of β2-microglobulin was 10 pg/ml in the sample (3 times the SD of the “zero” control; FIG. 3). A plateau in the signal was reached between 10 ng/ml and 100 ng/ml of β2-microglobulin.

A specific ELISA to measure the free β2-microglobulin in tetramer and monomer samples was developed. The accuracy of the assay was evaluated in dilution and spiking experiments, using previously defined conditions, and the content of free β2-microglobulin in 4 different monomer samples as well as 3 different tetramer samples was examined (Tables IX and X).

TABLE IX
Serial two fold dilution were carried out
and analyzed with the ELISA assay

| Sam- | Mean | OD | [b2m] | Dilution | Free b2m | Mean | |
|---|---|---|---|---|---|---|---|
| ple | OD | CV % | μg/ml | Factor | (μg/ml) | [b2m] | CV % |
| M00- | 0.85 | 3.60 | 1.42 | 10000 | 14.16 | 14.16 | 0.34% |
| 100 | 0.43 | 3.28 | 0.71 | 20000 | 14.20 | | |
| | 0.22 | 2.86 | 0.35 | 40000 | 14.11 | | |
| M00- | 0.97 | 0.29 | 1.63 | 10000 | 16.28 | 17.29 | 5.08% |
| 101 | 0.54 | 3.83 | 0.89 | 20000 | 17.74 | | |
| | 0.28 | 4.85 | 0.45 | 40000 | 17.86 | | |
| M00- | 0.85 | 2.58 | 1.42 | 10000 | 14.21 | 14.49 | 1.67% |
| 110 | 0.44 | 1.44 | 0.73 | 20000 | 14.60 | | |
| | 0.23 | 0.31 | 0.37 | 40000 | 14.65 | | |
| M00- | 0.84 | 3.70 | 1.41 | 10000 | 14.10 | 14.32 | 1.39% |
| 111 | 0.44 | 1.30 | 0.72 | 20000 | 14.37 | | |
| | 0.23 | 1.24 | 0.36 | 40000 | 14.48 | | |
| M00- | 0.22 | 0.98 | 0.34 | 10000 | 3.44 | 3.38 | 5.17% |
| 116 | 0.12 | 3.57 | 0.18 | 20000 | 3.52 | | |
| | 0.06 | 7.95 | 0.08 | 40000 | 3.19 | | |
| M00- | 0.27 | 4.19 | 0.43 | 10000 | 4.34 | 4.11 | 5.28% |
| 117 | 0.14 | 5.21 | 0.21 | 20000 | 4.10 | | |
| | 0.07 | 5.83 | 0.10 | 40000 | 3.90 | | |
| M00- | 0.26 | 1.08 | 0.42 | 10000 | 4.20 | 4.12 | 5.13% |
| 123 | 0.13 | 0.55 | 0.19 | 20000 | 3.88 | | |
| | 0.08 | 13.55 | 0.11 | 40000 | 4.28 | | |

TABLE X
Monomer and tetramer samples were spiked with a constant concentration of
β2-microglobulin, and serial two fold dilution were examined using the ELISA

| | | | | | | | [b2m] | |
|---|---|---|---|---|---|---|---|---|
| | | | | Free | | | without | |
| | Mean | OD | Dilution | b2m | Mean | | spiking | % |
| Sample | OD | CV % | Factor | (μg/ml) | [b2m] | CV % | (μg/ml) | Recovery |
| M00-100 | 1.23 | 2.58% | 10000 | 21.52 | 22.50 | 3.85% | 12.49 | 98% |
| | 0.66 | 0.96% | 20000 | 22.83 | | | | |
| | 0.34 | 1.04% | 40000 | 23.15 | | | | |
| M00-101 | 1.41 | 2.35% | 10000 | 24.67 | 25.62 | 3.46% | 19.18 | 86% |
| | 0.74 | 1.24% | 20000 | 25.75 | | | | |
| | 0.39 | 0.73% | 40000 | 26.43 | | | | |
| M00-110 | 1.16 | 2.01% | 10000 | 20.21 | 20.62 | 1.74% | 11.77 | 92% |
| | 0.60 | 1.64% | 20000 | 20.80 | | | | |
| | 0.31 | 3.20% | 40000 | 20.86 | | | | |
| M00-111 | 1.26 | 0.95% | 10000 | 21.97 | 22.15 | 2.92% | 12.18 | 97% |
| | 0.66 | 0.75% | 20000 | 22.86 | | | | |
| | 0.32 | 4.21% | 40000 | 21.60 | | | | |
| M00-116 | 0.82 | 3.09% | 10000 | 14.28 | 14.03 | 1.68% | 4.09 | 96% |
| | 0.41 | 5.34% | 20000 | 14.01 | | | | |
| | 0.21 | 5.41% | 40000 | 13.81 | | | | |
| M00-117 | 0.88 | 5.20% | 10000 | 15.36 | 15.04 | 2.54% | 5.39 | 94% |
| | 0.44 | 2.08% | 20000 | 15.14 | | | | |
| | 0.22 | 0.32% | 40000 | 14.62 | | | | |
| M00-123 | 0.88 | 0.32% | 10000 | 15.35 | 15.04 | 2.37% | 5.54 | 93% |
| | 0.44 | 0.64% | 20000 | 15.12 | | | | |
| | 0.22 | 1.92% | 40000 | 14.65 | | | | |
| Buffer + | 0.62 | 0.11% | 10000 | 10.62 | 10.55 | 2.94% | N/A | N/A |
| b2m | 0.32 | 0.88% | 20000 | 10.82 | | | | |
| | 0.16 | 6.27% | 40000 | 10.21 | | | | |

Competition Assay to Measure Free β2-microglobulin

A commercially available kit (competitive assay) to measure β2-microglobulin is available (Immunotech). This kit allows for an immunoassay competition assay that uses the β2-microglobulin directly conjugated to alkaline phosphatase and measures the total β2-microglobulin in a sample. However, the characteristics of the B1G6 mAb make it impossible to use the commercially available kit to measure the free β2-microglobulin in an MHC tetramer or MHC monomer sample. Accordingly, the competition assay disclosed herein utilized the reagents of the commercial kit, including the alkaline phosphatase-conjugated β2-microglobulin and the β2-microglobulin standards, except that the B1G6 antibody in the commercial kit was replaced with the C21.48A antibody, which specifically binds only free β2-microglobulin. Although the competition assay was expected to provide less sensitive results than the sandwich assay (though greater sensitivity than the SEC assay), the competition assay provides the advantage that is does not require the numerous dilutions that were necessary to perform the sandwich ELISA. The competition assay procedure is shown in Table XI.

TABLE XI
Summary of Competition Assay Procedure

| Step 1 | Step 2 | Step 3 | Step 4 |
|---|---|---|---|
| To well coated with | Aspirate | Dispense 200 | Add 50 μl of |
| monoclonal antibody | Rinse 3 times | μl/well of | stop solution |
| C21.48A add 10 μl of | with 300 μl of | pNPP | NaOH 1N |
| standard or sample and | wash solution | substrate | Read at |
| 200 μl of | | Incubate 30 | 405 nm |
| β2microglobulin-alkaline | | min with | |
| phosphatase conjugated | | shaking | |
| Incubate 90 min | | At 18-25° | |
| With shaking | | | |
| At 18-25° C. | | | |

Optimization of C21.48A mAb Concentration in the Microtiter Plates

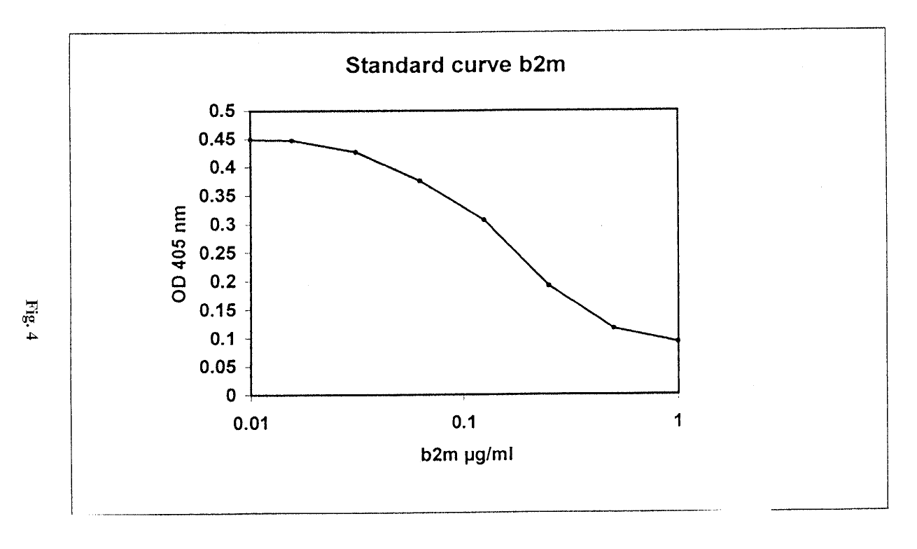

To determine the optimal concentration of C21.48A antibody for the competition assay, several wells of a 96 well plate were coated with different quantities of C21.48A mAb. After saturation of the wells with PBS/3% BSA, the alkaline phosphatase-β2-microglobulin conjugate was added. The signal was observed to saturate with increasing concentrations C21.48A mAb, and was maximal at 5 μg/ml of antibody. Based on this result, plates were coated with 5 μg/ml of C21.48A mAb for experiments to determine the effect of the free β2-microglobulin, and the sensitivity of the assay. The standard curve range was determined to be between 1 μg/ml and 0.07 μg/ml of β2-microglobulin (FIG. 4).

The accuracy of the assay was evaluated in dilution and spiking experiments. In a first experiment, free β2-microglobulin containing MHC monomers and MHC tetramers were spiked with different concentrations of recombinant β2-microglobulin, incubated 1 hr at RT, and analyzed by the competition assay. In a second experiment, MHC monomer and tetramer samples were serially diluted with PBS/1% BSA/10 mM NaN₃ and analyzed by the competition assay. In both experiments the recovery was excellent (see Tables XII and XIII).

TABLE XII
Serial two fold dilution analysis using the competition assay

| | | | Free | | | Total | | |
|---|---|---|---|---|---|---|---|---|
| | Mean | OD | β2m | | Dilution | Free β2m | | |
| Sample | OD | CV % | (μg/ml) | CV % | Factor | (μg/ml) | Mean | CV % |
| Monomer | 0.090 | 10.3% | 1.85 | 63.8 | 1/8 | 14.78 | | |
| M00-100 | 0.108 | 0.0% | 0.75 | 0 | 1/16 | 12 | | |
| | 0.145 | 5.4% | 0.47 | 7.1 | 1/32 | 15.1 | | |
| | 0.267 | 0.5% | 0.23 | 0.7 | 1/64 | 14.49 | 14.80 | 2.9% |
| Monomer | 0.093 | 2.3% | 1.17 | 9.2 | 1/8 | 9.33 | | |
| M00-101 | 0.108 | 2.6% | 0.75 | 5.5 | 1/16 | 12.02 | | |
| | 0.147 | 1.9% | 0.46 | 2.5 | 1/32 | 14.72 | | |
| | 0.253 | 0.6% | 0.24 | 0.7 | 1/64 | 15.48 | 15.1 | 3.6% |
| Monomer | 0.092 | 0.8% | 1.21 | 3.3 | 1/8 | 9.71 | | |
| M00-110 | 0.105 | 3.4% | 0.81 | 7.8 | 1/16 | 12.95 | | |
| | 0.148 | 2.4% | 0.46 | 3.1 | 1/32 | 14.66 | | |
| | 0.241 | 3.8% | 0.26 | 4.5 | 1/64 | 16.47 | 15.6 | 8.2% |
| Monomer | 0.094 | 0.8% | 1.11 | 2.8 | 1/8 | 8.89 | | |
| M00-111 | 0.111 | 0.0% | 0.71 | 0 | 1/16 | 11.35 | | |
| | 0.158 | 1.8% | 0.42 | 2.2 | 1/32 | 13.44 | | |
| | 0.271 | 1.0% | 0.22 | 1.3 | 1/64 | 14.22 | 13.83 | 4.0% |
| Monomer | 0.088 | 4.0% | 1.62 | 27.5 | 1/8 | 12.99 | | |
| M00-015 | 0.100 | 2.1% | 0.91 | 5.9 | 1/16 | 14.62 | | |
| | 0.134 | 2.6% | 0.52 | 3.8 | 1/32 | 16.78 | | |
| | 0.230 | 0.3% | 0.27 | 0.4 | 1/64 | 17.38 | 17.08 | 2.5% |
| Tetramer | 0.147 | 3.8% | 0.45 | 4.9 | 1/8 | 3.62 | | |
| M00-116 | 0.280 | 3.8% | 0.21 | 4.9 | 1/16 | 3.42 | | |
| | 0.394 | 7.5% | 0.12 | 17.6 | 1/32 | 3.88 | | |
| | 0.463 | 1.1% | 0.07 | 6 | 1/64 | 4.46 | 3.64 | 6.3% |
| Tetramer | 0.128 | 16.1% | 0.58 | 24.8 | 1/8 | 4.67 | | |
| M00-117 | 0.239 | 6.8% | 0.26 | 8.1 | 1/16 | 4.16 | | |
| | 0.350 | 5.9% | 0.15 | 10.1 | 1/32 | 4.92 | | |
| | 0.451 | 0.2% | 0.08 | 0.7 | 1/64 | 5.09 | 4.58 | 8.5% |
| Tetramer | 0.149 | 3.4% | 0.46 | 4.4 | 1/8 | 3.7 | | |
| M00-123 | 0.246 | 3.4% | 0.25 | 4.1 | 1/16 | 4.01 | | |
| | 0.376 | 1.7% | 0.13 | 3.4 | 1/32 | 4.3 | | |
| | 0.445 | 1.6% | 0.08 | 6.5 | 1/64 | 5.37 | 4.00 | 7.5% |

TABLE XIII
Monomer and tetramer samples spiked with a constant concentration of
β2microglobulin. Serial two fold dilution analysis using the competition assay.

| | | | Free | | | Free | | | | % Recovery |
|---|---|---|---|---|---|---|---|---|---|---|
| | Mean | OD | b2m | | Dilution | b2m | | | β2m | measured/ |
| Sample | OD | CV % | (μg/ml) | CV % | Factor | (μg/ml) | Mean | CV % | spiked | expected |
| Monomer | 0.27 | 16.9% | 0.21 | 22.9 | Not | 13.27 | | | | |
| M00-100 | | | | | spiked | | | | | |
| | | | | | 1/64* | | | | | |
| | 0.11 | 0.0% | 0.81 | 0 | 1/20 | 16.26 | | | | |
| | 0.14 | 1.5% | 0.51 | 2.5 | 1/40 | 20.3 | | | | |
| | 0.25 | 0.6% | 0.22 | 0.7 | 1/80 | 17.8 | | | | |
| | 0.40 | 0.7% | 0.11 | 1.6 | 1/160 | 17.19 | 18.4 | 8.9% | 5.2 | 92% |
| Monomer | 0.27 | 1.1% | 0.21 | 1.4 | Not | 13.4 | | | | |
| M00-101 | | | | | spiked | | | | | |
| | | | | | 1/64* | | | | | |
| | 0.11 | 2.0% | 0.87 | 5.5 | 1/20 | 17.33 | | | | |
| | 0.13 | 4.4% | 0.56 | 7.5 | 1/40 | 22.34 | | | | |
| | 0.22 | 16.6% | 0.28 | 21.1 | 1/80 | 22.17 | | | | |
| | 0.34 | 2.9% | 0.15 | 4.8 | 1/160 | 23.39 | 22.6 | 2.9% | 9.2 | 113% |
| Monomer | 0.27 | 13.8% | 0.21 | 18.5 | Not | 13.56 | | | | |
| M00-110 | | | | | spiked | | | | | |
| | | | | | 1/64* | | | | | |
| | 0.10 | 2.0% | 0.91 | 6 | 1/20 | 18.3 | | | | |
| | 0.13 | 0.5% | 0.53 | 0.9 | 1/40 | 21.29 | | | | |
| | 0.21 | 3.4% | 0.28 | 4.3 | 1/80 | 22.61 | | | | |
| | 0.36 | 4.6% | 0.14 | 8.1 | 1/160 | 21.69 | 21.9 | 3.1% | 8.3 | 109% |
| Monomer | 0.29 | 8.8% | 0.19 | 12.4 | Not | 12.01 | | | | |
| M00-111 | | | | | spiked | | | | | |
| | | | | | 1/64* | | | | | |
| | 0.10 | 2.9% | 1.06 | 10.1 | 1/20 | 21.19 | | | | |
| | 0.13 | 2.7% | 0.55 | 4.6 | 1/40 | 22.15 | | | | |
| | 0.21 | 4.1% | 0.29 | 5.2 | 1/80 | 23.03 | | | | |
| | 0.38 | 1.7% | 0.12 | 3.3 | 1/160 | 19.49 | 21.6 | 8.5% | 9.5 | 108% |
| Tetramer | 0.43 | 2.0% | 0.09 | 5.3 | Not | 2.92 | | | | |
| M00-116 | | | | | spiked | | | | | |
| | | | | | 1/32* | | | | | |
| | 0.14 | 1.6% | 0.52 | 2.5 | 1/20 | 10.39 | | | | |
| | 0.21 | 21.7% | 0.3 | 27.6 | 1/40 | 11.84 | | | | |
| | 0.38 | 6.8% | 0.12 | 13.2 | 1/80 | 9.74 | | | | |
| | 0.49 | 6.8% | 0.05 | 38.5 | 1/160 | 8.59 | 10.1 | 16.4% | 7.1 | 101% |
| Tetramer | 0.38 | 16.6% | 0.12 | 32.7 | Not | 3.89 | | | | |
| M00-117 | | | | | spiked | | | | | |
| | | | | | 1/32* | | | | | |
| | 0.15 | 3.8% | 0.46 | 5.7 | 1/20 | 9.18 | | | | |
| | 0.21 | 1.0% | 0.28 | 1.3 | 1/40 | 11.06 | | | | |
| | 0.37 | 4.2% | 0.13 | 7.9 | 1/80 | 10.24 | | | | |
| | 0.48 | 3.1% | 0.06 | 15.2 | 1/160 | 9.37 | 10.2 | 8.3% | 6.3 | 102% |
| Tetramer | 0.40 | 0.5% | 0.11 | 1.2 | Not | 3.43 | | | | |
| M00-123 | | | | | spiked | | | | | |
| | | | | | 1/32* | | | | | |
| | 0.13 | 4.8% | 0.55 | 8.2 | 1/20 | 10.96 | | | | |
| | 0.29 | 3.7% | 0.19 | 5.2 | 1/40 | 7.54 | | | | |
| | 0.44 | 2.0% | 0.09 | 5.5 | 1/80 | 6.97 | 8.5 | 25.4% | 3.5 | 85% |
| | 0.56 | 1.0% | Range | Range | 1/160 | Range | | | | |

Correlation between the Sandwich ELISA and the Competition Assay

The concentration of the free β2-microglobulin obtained with four MHC monomers and three MHC tetramers was compared using the sandwich assay and the competition assays. A very good correlation was obtained between the two assays, with a slope close to 1.

Standard (β2microglobulin Used in the Assay

An important aspect in the development of an immunometric assay is the selection of the molecule that should be used as standard. Two molecules were examined, including a recombinant β2-microglobulin (rβ2m), which was produced in E. coli and folded and purified, and a naturally occurring β2-microglobulin, which was purified from urine (Immunotech). The natural β2-microglobulin was stored freeze-dried and the rβ2m was stored liquid at −80° C. An advantage of using the natural β2-microglobulin is that the concentration is calibrated against an international standard molecule (WHO International Laboratory for Biological Standards) and is stored freeze-dried. However, the β2-microglobulin component of the MHC monomers and tetramers examined herein is the rβ2m. Accordingly, both β2-microglobulin species were examined and the results compared.

The amount of rβ2m in two different batches, Lot # M-00-0519 and Lot # M-00-153, which contain 690 and 614 μg/ml, respectively, of β2-microglobulin as measured by OD 280 nm, was examined. These concentrations were measured by OD at 280 nm and applying the coefficient of molar extinction of the β2-microglobulin. In parallel, the content of total proteins was determined using Coomassie Blue, with BSA (bovine serum albumin) as a standard. The results are shown in Table XIV.

TABLE XIV
Comparison two lots of β2-microglobulin

| | rb2m Lot | |
|---|---|---|
| | M-00-0519 | rb2m LotM-00-153 |
| Assay | (μg/ml) | (μg/ml) |
| OD 280 nm ext.coeff. [εm] = 1.56 | 690 | 614 |
| Competition assay | 908.8 ± 90.5 | 940.5 ± 78 |
| BIG6 mAb (Commercial kit) | | |
| Competition assay C21.48A mAb | 759 ± 32.5 | 630 ± 14.14 |
| ELISA C21.48A mAb solid phase | 1004.66 ± 77 | 1038.11 ± 79 |
| B1G6mAb as Tracer | | |
| Coomassie Blue | 878.96 ± 61 | 655.79 ± 32 |

The content of β2-microglobulin measured with the ELISA and the commercial kit (B1G6 mAb) was greater than that measured with the competition assay using either the C21.48A mAb, Coomassie Blue, or optical density. Thus, the assays that over measure the β2-microglobulin both use the B1G6 antibody. For the Lot M-00-153 of β2-microglobulin, the values obtained by OD 280, the competition assay using C21.48A, and the Coomassie Blue were very close. These differences cannot be explained by the purity of the proteins because the SDS-PAGE and gel filtration chromatography revealed a pure and homogenous molecule.

A major difference was observed when the B1G6 antibody was used as tracer or coated in a solid phase, suggesting that the B1G6 mAb better recognized the recombinant β2-microglobulin. However, when the level of the free β2-microglobulin in MHC monomer and tetramer samples was compared, both assays correlated well (y=1.0357×−03196; r²=0.9698). These results indicated that β2-microglobulin derived from the dissociation of the MHC monomer is correctly folded and has the same structure as the β2-microglobulin from urine, and is recognized equally well for both assays. The results also indicate that, when the β2-microglobulin is folded alone, the epitope recognized by the B1G6 antibody is folded slightly differently and, as a result, better recognized by the antibody.

When the antibodies were used in western blot assays, B1G6 recognized β2-microglobulin very well under reducing and non-reducing conditions, whereas the C21.48A mAb recognized both species of β2-microglobulin less. This result indicates that the epitope of the C21.48A is a conformational epitope, and the epitope recognized by B1G6 is a linear epitope that can be modified during the folding of the molecule. Such a result also explains why the assays using the B1G6 mAb over-estimate that amount of rβ2m.

Correlation of Competition and ELISA Assays with SEC

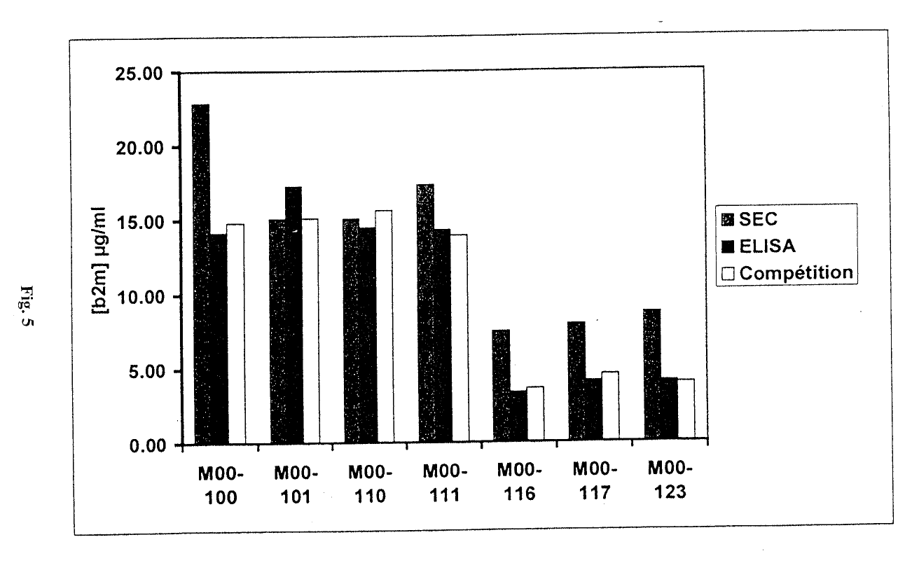

The results of measurements of the free β2-microglobulin described above were determined by SEC (size exclusion chromatography). To analyze the correlation between the level of β2-microglobulin determined by SEC with both immunoassays, the assays measuring the free β2-microglobulin of the MHC monomer and MHC tetramer samples, as well as samples containing different quantities of purified β2-microglobulin, were performed in parallel. A very good correlation was observed between the SEC and both EIAs. The slope of the curve after linear regression was close to 1 (ELISA/SEC: y=0.927; r²=0.8113; competition/SEC: y=0.6492; r²=0.849). However, the comparison for the lower values was not very good (FIG. 5).

One of the principal problems with the size exclusion chromatography is the sensitivity of the assay and the extremely high variation for the lower values. This error is due to the integration area under the peak, wherein a small variation in the integration has a strong effect in the quantification of the free β2-microglobulin. This effect can be observed clearly in FIG. 5. In this case, the variability between the immunoassays and the SEC is higher at the lower values; there was good correlation, however, for the highest values. These results indicate that an EIA as disclosed herein can be used to obtain accurate measurements of free β2-microglobulin.

Conclusions

Two different anti-β2-microglobulin monoclonal antibodies and two different immunometric assays to measure free β2-microglobulin in MHC monomer and MHC tetramer samples were characterized. The anti-β2-microglobulin B1G6 mAb and anti-β2-microglobulin C21.48A mAb are available (Immunotech; BIODESIGN catalogue). The specificity of both antibodies was clearly demonstrated (Liabeuf et al., supra, 1981; Devaux et al., supra, 1990), and the reported observation fits well with the present results; for example, the biotinylated monomers did not bind to the C21.48A coated plates, whereas the biotinylated β2-microglobulin bound well to the same plates.

The characteristics of these antibodies were utilized to design a specific ELISA and a specific competition assay for measuring free β2-microglobulin such as that derived from the dissociation of MHC monomer and MHC tetramers. The ELISA assay used the C21.48A mAb coated in solid phase, and the B1G6 mAb conjugated to the peroxidase as tracer; the competition assay used the C21.48A mAb coated in a solid phase and β2-microglobulin conjugated to the alkaline phosphatase as tracer.

Both immunoassays permitted precise and reproducible measurements of free β2-microglobulin, as demonstrated by the dilution and the spiking experiments. The sensitivity f the ELISA was calculated to be 10 pg/ml and the sensitivity of the competition assay was determined to be 0.5 μg/ml, and there was a very good correlation between the assays. The immunoassays also correlated well with the size exclusion chromatography for the higher values; however, for the lower values of free β2-microglobulin, the gel filtration chromatography did not fit well with the assays.

A comparison between the rβ2m produced in E. coli and folded alone and the natural β2-microglobulin also revealed a difference in the recognition by the B1G6 antibody. However, the rβ2m derived from the dissociation of the monomer was likely folded similarly to the natural β2-microglobulin because the ELISA and competition assay give similar results. These findings indicate that the rβ2m, once calibrated against the natural β2-microglobulin, can be used as a standard.

### EXAMPLE 3

### Validation of Enzyme Immunoassay

Four different parameters that can influence an immunoassay of the invention were examined to validate the immunoassays: 1) the anti-β2-microglobulin coated microtiter plates; 2) the operator; 3) the plate reader; and the temperature for performing the enzymatic reaction. These four parameters were taken into account to design six groups (Table XV) that allow a determination of the precision and the reproducibility of the assays, as well as the accuracy and the robustness. The experiences of the operators regarding the ELISA and EIA tests was A>B>C. The results of the validation studies are shown in Tables XVI to XVIII. Tables XIX summarizes the MHC monomers and MHC tetramers, and plates used in the validation studies.

TABLE XV
Samples used for validation studies

| | | Date of manufacturing |
|---|---|---|
| Sample | Lot No. | or expiration |
| Monomer HLA-A*0201/HIVpol | M00-102 | Manufacturing 16/11/00 |
| Monomer HLA-A*0201/Mart1 | M00-045 | Manufacturing 14/08/00 |
| Monomer HLA-A*0201/Mart1 | M00-044 | Manufacturing 11/08/00 |
| Tetramer Mart1/+CD8 | M00-127 | Expiration 17/05/01 |
| Tetramer HIVgag/+CD8 | M00-130 | Expiration 21/05/01 |
| Tetramer HIVpol/+CD8 | M00-124 | Expiration 16/05/01 |

TABLE XVI
Microtiter plates used for validation studies

| Lot No. | Plates | Date of validation |
|---|---|---|
| D00-031 | Microtiter plates anti-β2m C21.48A mAb | 19/01/01 |
| D00-032 | Microtiter plates anti-β2m C21.48A mAb | 23/01/01 |
| D00-033 | Microtiter plates anti-β2m C21.48A mAb | 23/01/01 |

TABLE XVII
Summary of groups for validating immunoassays

| Group | I | II | III | IV | V | VI |
|---|---|---|---|---|---|---|
| Operator | A | A | B | B | C | C |
| Day | 1 | 1 | 2 | 2 | 3 | 3 |
| Plate | D00-031 | D00-032 | D00-032 | D00-033 | D00-031 | D00-033 |
| Temperature of | 21° C. | 21° C. | 28° C. | 28° C. | 31° C. | 31° C. |
| enzymatic activity | | | | | | |
| reaction | | | | | | |
| Number of samples | 6 | 6 | 6 | 6 | 6 | 6 |
| Reader | 1 | 1 | 2 | 2 | 3 | 3 |

TABLE XVIII
Results

| | I | | | | | | | | | | | |
|---|---|---|---|---|---|---|---|---|---|---|---|---|
| Operator | Mono | II | I | II | I | II | I | II | I | II | I | II |
| A Day 1 | M00- | Mono | Mono | Mono | Mono | Mono | Tetra | Tetra | Tetra | Tetra | Tetra | Tetra |
| Group | 044 | M00-044 | M00-045 | M00-045 | M00-102 | M00-102 | M00-124 | M00-124 | M00-127 | M00-127 | M00-130 | M00-130 |
| Plate | D031 | D032 | D031 | D032 | D031 | D032 | D031 | D032 | D031 | D032 | D031 | D032 |
| 1 | 7.68 | 7.04 | 5.76 | 5.76 | 7.68 | 7.04 | 3.52 | 3.2 | 3.2 | 2.88 | 5.44 | 5.76 |
| 2 | 7.68 | 7.68 | 7.04 | 5.76 | 8.96 | 7.04 | 3.84 | 3.2 | 3.52 | 3.2 | 6.08 | 6.08 |
| 3 | 8.96 | 8.32 | 7.04 | 5.76 | 9.6 | 7.68 | 4.16 | 3.52 | 3.52 | 3.52 | 6.4 | 6.08 |
| 4 | 8.96 | 8.32 | 7.68 | 6.4 | 9.6 | 7.68 | 4.16 | 3.52 | 3.84 | 3.2 | 6.72 | 6.4 |
| 5 | 8.96 | 7.68 | 7.68 | 5.76 | 8.32 | 7.68 | 4.16 | 3.52 | 3.84 | 3.52 | 6.72 | 6.08 |
| 6 | 8.96 | 8.32 | 7.68 | 6.4 | 9.6 | 8.32 | 3.84 | 3.52 | 3.84 | 3.52 | 6.72 | 6.4 |
| 7 | 9.6 | 7.68 | 7.04 | 6.4 | 8.96 | 7.68 | 3.52 | 3.2 | 4.16 | 3.84 | 6.4 | 6.08 |
| 8 | 9.6 | | 7.04 | 5.76 | 8.96 | 7.68 | 3.52 | 3.52 | 3.84 | 3.84 | 6.72 | 6.4 |
| 9 | 8.96 | 8.32 | 6.4 | 5.76 | 8.96 | 7.68 | 3.2 | 3.2 | 4.16 | 3.52 | 6.4 | 6.08 |
| 10 | 8.32 | 7.68 | 6.4 | 5.76 | 7.68 | 7.68 | 3.52 | 3.2 | 3.52 | 3.52 | 6.08 | 5.76 |
| Mean | 8.768 | 7.89 | 6.976 | 5.952 | 8.832 | 7.616 | 3.744 | 3.36 | 3.744 | 3.456 | 6.368 | 6.112 |
| Mean − | 0.44 | −0.43 | 0.53 | −0.50 | 0.77 | −0.45 | −0.11 | −0.49 | 0.17 | −0.12 | 0.23 | −0.02 |
| mean of | | | | | | | | | | | | |
| the mean | | | | | | | | | | | | |
| SD | 0.68 | 0.45 | 0.64 | 0.31 | 0.73 | 0.36 | 0.34 | 0.17 | 0.30 | 0.29 | 0.41 | 0.24 |
| CV | 7.73 | 5.73 | 9.12 | 5.19 | 8.23 | 4.77 | 9.05 | 5.02 | 8.11 | 8.51 | 6.47 | 3.86 |
| Variance | 0.46 | 0.20 | 0.41 | 0.10 | 0.53 | 0.13 | 0.11 | 0.03 | 0.09 | 0.09 | 0.17 | 0.06 |
| N | 10 | 9 | 10 | 10 | 10 | 10 | 10 | 10 | 10 | 10 | 10 | 10 |
| Operator | III | | | | | | | | | | | |
| B | Mono | IV | III | IV | III | IV | III | IV | III | IV | III | IV |
| Day 2 | M00- | Mono | Mono | Mono | Mono | Mono | Tetra | Tetra | Tetra | Tetra | Tetra | Tetra |
| Group | 044 | M00-044 | M00-045 | M00-045 | M00-102 | M00-102 | M00-124 | M00-124 | M00-127 | M00-127 | M00-130 | M00-130 |
| Plate | D032 | D033 | D032 | D033 | D032 | D033 | D032 | D033 | D032 | D033 | D032 | D033 |
| 1 | 7.04 | | 5.12 | 7.04 | 7.04 | 8.32 | 3.2 | 4.16 | 3.2 | 3.84 | 5.12 | 6.4 |
| 2 | 7.04 | 8.32 | 4.48 | 6.4 | 7.04 | 7.04 | 3.52 | 3.52 | 3.2 | 3.2 | 5.44 | 5.12 |
| 3 | 7.04 | 8.32 | 5.76 | 5.76 | 7.04 | 7.68 | 3.52 | 3.52 | 3.2 | 3.52 | 5.44 | 5.44 |
| 4 | 7.04 | 8.32 | 5.12 | 5.76 | 7.04 | 7.68 | 3.2 | 3.52 | 3.2 | 3.2 | 6.72 | 5.12 |
| 5 | 6.4 | 8.96 | 5.12 | 6.4 | 7.04 | 8.96 | 3.52 | 3.84 | 3.2 | 3.52 | | 5.76 |
| 6 | 7.68 | 8.96 | 5.12 | 6.4 | 7.04 | 7.68 | 3.52 | 3.84 | 3.52 | 3.84 | | 5.76 |
| 7 | 6.4 | 8.96 | 5.12 | 6.4 | 7.04 | 8.32 | 3.52 | 3.84 | 3.52 | 3.84 | 5.76 | 6.08 |
| 8 | 7.04 | 8.96 | 5.12 | 6.4 | 7.04 | 8.32 | 3.52 | 4.16 | 3.52 | 4.16 | 5.44 | 6.08 |
| 9 | 7.04 | 9.6 | 5.12 | 7.04 | 7.04 | 8.32 | 3.52 | 4.48 | 3.52 | 4.16 | 5.44 | 6.4 |
| 10 | 5.76 | 8.32 | 4.48 | 5.76 | 6.4 | 8.32 | 3.2 | 3.84 | 3.2 | 4.16 | 5.44 | 6.4 |
| Mean | 6.85 | 8.75 | 5.056 | 6.336 | 6.976 | 8.064 | 3.424 | 3.872 | 3.328 | 3.744 | 5.60 | 5.856 |
| Mean − | −1.48 | 0.42 | −1.39 | −0.11 | −1.09 | 0.00 | −0.43 | 0.02 | −0.25 | 0.17 | −0.54 | −0.28 |
| mean of | | | | | | | | | | | | |
| the mean | | | | | | | | | | | | |
| SD | 0.53 | 0.45 | 0.36 | 0.47 | 0.20 | 0.54 | 0.15 | 0.32 | 0.17 | 0.37 | 0.48 | 0.50 |
| CV | 7.69 | 5.17 | 7.19 | 7.45 | 2.90 | 6.69 | 4.51 | 8.22 | 4.97 | 9.91 | 8.64 | 8.56 |
| Variance | 0.28 | 0.20 | 0.13 | 0.22 | 0.04 | 0.29 | 0.02 | 0.10 | 0.03 | 0.14 | 0.23 | 0.25 |
| N | 10 | 9 | 10 | 10 | 10 | 10 | 10 | 10 | 10 | 10 | 8 | 10 |
| Operator | V | | | | | | | | | | | |
| 3 | Mono | VI | V | VI | V | VI | V | VI | V | VI | V | VI |
| Day 3 | M00- | Mono | Mono | Mono | Mono | Mono | Tetra | Tetra | Tetra | Tetra | Tetra | Tetra |
| Group | 044 | M00-044 | M00-045 | M00-045 | M00-102 | M00-102 | M00-124 | M00-124 | M00-127 | M00-127 | M00-130 | M00-130 |
| Plate | D031 | D033 | D031 | D033 | D031 | D033 | D031 | D033 | D031 | D033 | D031 | D033 |
| 1 | 8.832 | 8.192 | 9.344 | 7.36 | 8.896 | 8.512 | 5.728 | 4.128 | 4.416 | 3.264 | 7.808 | 6.656 |
| 2 | 9.216 | 9.536 | 7.488 | 7.808 | 8.896 | 9.216 | 4.288 | 4.64 | 3.776 | 3.808 | 5.952 | 7.008 |
| 3 | 7.744 | 9.28 | 6.208 | 7.04 | 8.576 | 9.024 | 4.032 | 4.736 | 3.2 | 3.84 | 5.376 | 5.984 |
| 4 | 8.384 | 10.304 | 6.784 | 7.936 | 7.808 | 9.152 | 3.808 | 4.8 | 3.456 | 3.936 | 5.952 | 6.976 |
| 5 | 8.128 | 9.152 | 6.72 | 7.296 | 7.04 | 8.256 | 4 | 4.608 | 3.648 | 3.872 | 5.76 | 6.432 |
| 6 | 8.576 | 9.28 | 7.424 | 8.064 | 7.744 | 8.96 | 3.936 | 4.672 | 3.232 | 4.128 | 6.208 | 7.008 |
| 7 | 7.552 | 9.088 | 6.656 | 7.424 | 8 | 8.512 | 3.68 | 3.744 | 3.168 | 4 | 5.632 | 6.368 |
| 8 | 8.32 | 9.664 | 7.168 | 7.104 | 8.512 | 9.088 | 4.352 | 4.544 | 3.456 | 3.712 | 5.76 | 6.496 |
| 9 | 9.088 | 8.704 | 7.04 | 6.336 | 8.448 | 8.96 | 4.544 | 3.808 | 3.488 | 3.68 | 6.144 | 6.112 |
| 10 | 9.024 | 8.96 | 6.976 | 5.504 | 7.296 | 8 | 4.736 | 4.384 | 2.688 | 3.232 | 8.768 | 6.4 |
| Mean | 8.49 | 9.22 | 7.18 | 7.19 | 8.12 | 8.77 | 4.31 | 4.41 | 3.45 | 3.75 | 6.34 | 6.54 |
| Mean − | 0.16 | 0.89 | 0.73 | 0.74 | 0.06 | 0.71 | 0.46 | 0.55 | −0.13 | 0.17 | 0.20 | 0.41 |
| mean of | | | | | | | | | | | | |
| the mean | | | | | | | | | | | | |
| SD | 0.57 | 0.57 | 0.85 | 0.77 | 0.65 | 0.42 | 0.60 | 0.38 | 0.45 | 0.29 | 1.08 | 0.36 |
| CV | 6.69 | 6.15 | 11.82 | 10.78 | 7.97 | 4.77 | 13.86 | 8.70 | 13.13 | 7.84 | 17.06 | 5.57 |
| Variance | 0.32 | 0.32 | 0.72 | 0.60 | 0.42 | 0.17 | 0.36 | 0.15 | 0.21 | 0.09 | 1.17 | 0.13 |
| N | 10 | 10 | 10 | 10 | 10 | 10 | 10 | 10 | 10 | 10 | 10 | 10 |
| Mean of | 8.33 | | 6.45 | | 8.06 | | 3.85 | | 3.58 | | 6.14 | |
| the mean | | | | | | | | | | | | |

Results

Reproducibility

Statistical Analysis.

A summary of the statistical analysis is shown in Table XIX. The homogeneity of the variances was tested using the Cochran test. When the Cochran criteria (C) was lower at the 5% threshold (C=0.368), all the variances were considered as homogenous and no values were rejected. When C was between thresholds 5% and 1%, the highest variance was considered as “suspicious.” When C was higher than at the 1% threshold, the highest variance was considered as “aberrant.” In the two last cases, several points in the group could be aberrant and the Dixon test was used to detect the aberrant values. Following this analysis, aberrant values were detected in the sample M00-044 of groups II and IV, respectively, as well as in the sample M00-130 of group III. The final results take into account these modifications. The Dixon test failed to identify an aberrant value within values of the sample M00-130 of group V, which have the highest variance. The mean of the Precision CV was 8.36% and the mean of the Reproducibility CV was 9.67%, among the six different samples analyzed. Both results are considered as excellent.

Accuracy

A. Dilution Experiments

The accuracy of the test was calculated by dilution and spiking experiments. Results are shown below in Tables XX to XXII, and are summarized in Table XXIII.

Statistical Analysis

Dilution experiments were carried out as independent dilutions and analyzed in triplicate. Four different dilutions were analyzed and three dilutions were taken into account for the statistical analysis. The homogeneity of the variances was tested following the Cochran test. The variances were considered as homogeneous. The mean of the Precision CV was 6.69% and the mean of the Reproducibility CV was 9.1% for the dilution studies, considering the six different samples analyzed (see Tables XXIV and XXV). Both results correspond to the criteria defined in the validation protocol.

TABLE XIX
Summary of statistical analysis

| Group | I | II | III | IV | V | VI |
|---|---|---|---|---|---|---|
| Operator | A | A | B | B | C | C |
| Day | 1 | 1 | 2 | 2 | 3 | 3 |
| Plate | D00-031 | D00-032 | D00-032 | D00-033 | D00-031 | D00-033 |
| Temperature | 21° C. | 21° C. | 28° C. | 28° C. | 31° C. | 31° C. |
| Reader | 1 | 1 | 2 | 2 | 3 | 3 |

| | | | | | | | Statistical analysis | | | | | |
|---|---|---|---|---|---|---|---|---|---|---|---|---|
| | Variance | Variance | Variance | Variance | Variance | Variance | C | Sr2 | Sg2 | SR2 | CV rep | CV repro |
| Monomer | 0.46 | 0.2 | 0.28 | 0.2 | 0.32 | 0.32 | 0.258 | 0.300 | 0.473 | 0.773 | 6.58% | 10.6% |
| M00-044 | | | | | | | | | | | | |
| N | 10 | 9 | 10 | 9 | 10 | 10 | | | | | | |
| Monomer | 0.41 | 0.1 | 0.13 | 0.22 | 0.72 | 0.6 | 0.330 | 0.363 | 0.457 | 0.820 | 9.35% | 14.0% |
| M00-045 | | | | | | | | | | | | |
| N | 10 | 10 | 10 | 10 | 10 | 10 | | | | | | |
| Monomer | 0.53 | 0.13 | 0.04 | 0.29 | 0.42 | 0.17 | 0.335 | 0.263 | 0.286 | 0.549 | 6.36% | 9.2% |
| M00-102 | | | | | | | | | | | | |
| N | 10 | 10 | 10 | 10 | 10 | 10 | | | | | | |
| Tetramer | 0.11 | 0.03 | 0.02 | 0.1 | 0.36 | 0.15 | 0.468 | 0.128 | 0.046 | 0.174 | 9.30% | 10.8% |
| M00-124 | | | | | | | | | | | | |
| N | 10 | 10 | 10 | 10 | 10 | 10 | | | | | | |
| Tetramer | 0.09 | 0.09 | 0.03 | 0.14 | 0.21 | 0.09 | 0.323 | 0.108 | 0.081 | 0.026 | 9.20% | 4.6% |
| M00-127 | | | | | | | | | | | | |
| N | 10 | 10 | 10 | 10 | 10 | 10 | | | | | | |
| Tetramer | 0.17 | 0.06 | 0.23 | 0.2 | 1.17 | 0.13 | 0.597 | 0.330 | −0.04 | 0.290 | 9.37% | 8.8% |
| M00-130 | | | | | | | | | | | | |
| N | 10 | 10 | 8 | 10 | 10 | 10 | | | | | | |
| Mean | | | | | | | | | | | 8.36% | 9.67% |

Threshold of Cochran Test at 5% level. C(0.05, n = 10, k = 6) = 0.368;
Threshold of Cochran Test at 1% level. C(0.01, n = 10, k = 6) = 0.423.

B. Spiking Experiments.

Different samples were spiked with a final β2-microglobulin concentration at 5μg/ml. The samples were incubated at RT (23° C.) during 1 hr, then diluted and assayed for β2-microglobulin content with the EIA test. Group V was excluded from the statistical analysis because a major problem occurred with the automatic washer and with the automatic multi pipette, generating a high background, aberrant values and high CV in the plate.

The % of recovery for each sample analyzed in the different groups is shown in Table XXVI. The unspiked sample was diluted and the level of β2-microglobulin was determined. At the same time, the monomer or tetramer sample was spiked and diluted, and analyzed. A sample containing only buffer spiked with the β2-microglobulin also was included in the assay. This study allowed a determination of the exact concentration in β2-microglobulin spiked in the samples; % recovery was calculated as follows:

$$[ \frac{[ μg / ml of β 2 m measured in spiked sample ]}{[ μm / ml of β 2 m in non \text{-} spiked sample ] + [ μg / ml of β 2 m of spiked buffer ] } ] * 100$$

The Cochran test showed that the variances were homogenous {C=0.28 for C(0.05)=0.507 and C(0.01)=0.588}. The CV of repeatability was calculated to be 5.89 and the CV of reproducibility was 7.5, respectively. Both values were excellent.

| | |
|---|---|
| Mean of recovery | 93.1 |
| SD | 13.7 |
| CV | 14.7 |
| n | 35 |

TABLE XX
Results of dilution experiments

| Group I | Plate D00-031 | | | | | | Group II | Plate D00-032 | | | | | |
|---|---|---|---|---|---|---|---|---|---|---|---|---|---|
| Sample | Doses | % CV | Dilution | Doses | Mean | % CV | Doses | % CV | Dilution | Doses | Mean | % CV | Ratio 32/31 |
| Monomer | 0.75 | 5.6 | 8 | 6 | 8.0 | 6.9% | 0.68 | 3.9 | 8 | 5.44 | 7.0 | 4.5% | 0.88 |
| M00-044 | 0.46 | 4.5 | 16 | 7.36 | | | 0.42 | 3.1 | 16 | 6.72 | | | |
| | 0.26 | 3.6 | 32 | 8.32 | | | 0.23 | 5.1 | 32 | 7.36 | | | |
| | 0.13 | 2 | 64 | 8.32 | | | 0.11 | 8.5 | 64 | 7.04 | | | |
| Monomer | 0.66 | 2.1 | 8 | 5.28 | 5.4 | 5.9% | 0.58 | 2.1 | 8 | 4.64 | 4.8 | 8.8% | 0.87 |
| M00-045 | 0.36 | 7.1 | 16 | 5.76 | | | 0.33 | 10.2 | 16 | 5.28 | | | |
| | 0.17 | 4.2 | 32 | 5.44 | | | 0.14 | 6.1 | 32 | 4.48 | | | |
| | 0.08 | 8.7 | 64 | 5.12 | | | 0.07 | 6.2 | 64 | 4.48 | | | |
| Monomer | 0.83 | 6.1 | 8 | 6.64 | 7.4 | 13.0% | 0.77 | 4.1 | 8 | 6.16 | 6.9 | 7.1% | 0.94 |
| M00-102 | 0.52 | 3.7 | 16 | 8.32 | | | 0.46 | 3.6 | 16 | 7.36 | | | |
| | 0.23 | 8.2 | 32 | 7.36 | | | 0.22 | 3.9 | 32 | 7.04 | | | |
| | 0.1 | 14.8 | 64 | 6.4 | | | 0.1 | 14.4 | 64 | 6.4 | | | |
| Tetramer | 0.55 | 9.7 | 8 | 4.4 | 4.2 | 3.3% | 0.54 | 1.7 | 8 | 4.32 | 3.9 | 10.2% | 0.93 |
| M00-124 | 0.26 | 5.3 | 16 | 4.16 | | | 0.25 | 11.3 | 16 | 4 | | | |
| | 0.13 | 3.5 | 32 | 4.16 | | | 0.11 | 5.7 | 32 | 3.52 | | | |
| | 0.08 | 9 | 64 | 5.12 | | | 0.08 | 17.2 | 64 | 5.12 | | | |
| Tetramer | 0.49 | 8.1 | 8 | 3.92 | 3.9 | 8.4% | 0.43 | 2 | 8 | 3.44 | 3.8 | 2.4% | 0.94 |
| M00-127 | 0.26 | 5 | 16 | 4.16 | | | 0.23 | 2.4 | 16 | 3.68 | | | |
| | 0.11 | 4 | 32 | 3.52 | | | 0.12 | 24.7 | 32 | 3.84 | | | |
| | 0.07 | 1.8 | 64 | 4.48 | | | 0.06 | 17.4 | 64 | 3.84 | | | |
| Tetramer | 0.77 | 3 | 8 | 6.16 | 6.4 | 3.2% | 0.68 | 2.2 | 8 | 5.44 | 5.8 | 6.9% | 1.00 |
| M00-130 | 0.41 | 5 | 16 | 6.56 | | | 0.39 | 9.4 | 16 | 6.24 | | | |
| | 0.2 | 12.4 | 32 | 6.4 | | | 0.23 | 29.9 | 32 | 7.36 | | | |
| | 0.09 | 13 | 64 | 5.76 | | | 0.09 | 8.4 | 64 | 5.76 | | | |
| | | | | | | 6.8% | | | | | | 7.69% | 0.93 |

TABLE XXI
Results of dilution experiments

| Group III | Plate D00-032 | | | | | | Group IV | Plate D00-033 | | | | | |
|---|---|---|---|---|---|---|---|---|---|---|---|---|---|
| Sample | Doses | % CV | Dilution | Doses | Mean | % CV | Doses | % CV | Dilution | Doses | Mean | % CV | Ratio 32/33 |
| Monomer | 0.9 | 3 | 8 | 7.18 | 8.5 | 11.7% | 0.84 | 5.3 | 8 | 6.73 | 9.0 | 13.7% | 1.05 |
| M00-044 | 0.46 | 1.1 | 16 | 7.38 | | | 0.47 | 2.8 | 16 | 7.56 | | | |
| | 0.29 | 0.6 | 32 | 9.28 | | | 0.31 | 1.3 | 32 | 9.81 | | | |
| | 0.14 | 2 | 64 | 8.87 | | | 0.15 | 1.8 | 64 | 9.52 | | | |
| Monomer | 0.7 | 2.7 | 8 | 5.58 | 5.9 | 6.0% | 0.69 | 7.3 | 8 | 5.49 | 6.4 | 3.7% | 1.09 |
| M00-045 | 0.36 | 0.8 | 16 | 5.78 | | | 0.39 | 2.1 | 16 | 6.2 | | | |
| | 0.2 | 2.1 | 32 | 6.27 | | | 0.21 | 3.9 | 32 | 6.65 | | | |
| | 0.1 | 6.2 | 64 | 6.65 | | | 0.1 | 14 | 64 | 6.31 | | | |
| Monomer | 0.93 | 9.9 | 8 | 7.44 | 9.0 | 2.4% | 0.88 | 2.8 | 8 | 7.01 | 9.4 | 4.9% | 1.04 |
| M00-102 | 0.55 | 8.1 | 16 | 8.85 | | | 0.55 | 6.9 | 16 | 8.86 | | | |
| | 0.29 | 2.3 | 32 | 9.26 | | | 0.3 | 4.2 | 32 | 9.44 | | | |
| | 0.14 | 1.5 | 64 | 8.95 | | | 0.15 | 6.2 | 64 | 9.77 | | | |
| Tetramer | 0.54 | 3.1 | 8 | 4.35 | 4.3 | 3.3% | 0.58 | 2.6 | 8 | 4.66 | 4.4 | 5.2% | 1.03 |
| M00-124 | 0.28 | 1.7 | 16 | 4.43 | | | 0.28 | 6.2 | 16 | 4.44 | | | |
| | 0.13 | 6.5 | 32 | 4.15 | | | 0.13 | 2.2 | 32 | 4.2 | | | |
| | 0.08 | 12.2 | 64 | 5.29 | | | 0.08 | 4 | 64 | 4.85 | | | |
| Tetramer | 0.48 | 2.9 | 8 | 3.87 | 3.8 | 3.4% | 0.5 | 3.3 | 8 | 4 | 4.1 | 4.0% | 1.06 |
| M00-127 | 0.23 | 4.4 | 16 | 3.68 | | | 0.24 | 1.4 | 16 | 3.92 | | | |
| | 0.12 | 11.1 | 32 | 3.93 | | | 0.13 | 5.2 | 32 | 4.23 | | | |
| | 0.07 | 9.7 | 64 | 4.69 | | | 0.09 | 6 | 64 | 5.64 | | | |
| Tetramer | 0.85 | 7.3 | 8 | 6.79 | 6.4 | 5.9% | 0.79 | 9.2 | 8 | 6.35 | 6.4 | 2.0% | 1.00 |
| M00-130 | 0.38 | 1.6 | 16 | 6.04 | | | 0.41 | 0.5 | 16 | 6.53 | | | |
| | 0.2 | 8.7 | 32 | 6.32 | | | 0.2 | 1.6 | 32 | 6.28 | | | |
| | 0.11 | 4.5 | 64 | 6.84 | | | 0.11 | 4.8 | 64 | 7.17 | | | |
| | | | | | | | | | | | | | 1.04 |

TABLE XXII
Results of dilution experiments

| Group V | Plate D00-031 | | | | | | Group VI | Plate D00-033 | | | | | |
|---|---|---|---|---|---|---|---|---|---|---|---|---|---|
| Sample | Doses | % CV | Dilution | Conc | Mean | CV | Doses | % CV | Dilution | Conc | Mean | CV | Ratio 31/33 |
| Monomer | 0.872 | 6.5 | 8 | 6.978 | 8.58 | 7.47 | 0.84 | 0.9 | 8 | 6.718 | 8.43 | 5.77 | 0.98 |
| M00-044 | 0.541 | 1.7 | 16 | 8.65 | | | 0.514 | 4.4 | 16 | 8.226 | | | |
| | 0.248 | 9.1 | 32 | 7.94 | | | 0.252 | 4.8 | 32 | 8.065 | | | |
| | 0.143 | 11.6 | 64 | 9.155 | | | 0.141 | 8.1 | 64 | 8.99 | | | |
| Monomer | 0.722 | 4.1 | 8 | 5.779 | 5.83 | 9 | 0.675 | 4.9 | 8 | 5.4 | 6.03 | 5.83 | 1.03 |
| M00-045 | 0.356 | 6.6 | 16 | 5.698 | | | 0.384 | 5 | 16 | 6.146 | | | |
| | 0.2 | 12 | 32 | 6.404 | | | 0.191 | 4.3 | 32 | 6.113 | | | |
| | 0.084 | 8.4 | 64 | 5.39 | | | 0.091 | 8.2 | 64 | 5.821 | | | |
| Monomer | 0.903 | 6.9 | 8 | 7.22 | 8.28 | 5.4 | 0.844 | 1.6 | 8 | 6.755 | 8.24 | 2.63 | 1.0 |
| M00-102 | 0.558 | 4.7 | 16 | 8.92 | | | 0.532 | 1.8 | 16 | 8.505 | | | |
| | 0.238 | 4.8 | 32 | 7.62 | | | 0.257 | 4.5 | 32 | 8.23 | | | |
| | 0.13 | 6.7 | 64 | 8.29 | | | 0.125 | 1.6 | 64 | 7.98 | | | |
| Tetramer | 0.559 | 5.2 | 8 | 4.74 | 3.92 | 11.7 | 0.543 | 1 | 8 | 4.34 | 4.03 | 3.3 | 1.03 |
| M00-124 | 0.231 | 11.5 | 16 | 3.69 | | | 0.243 | 4.5 | 16 | 3.89 | | | |
| | 0.132 | 6.6 | 32 | 4.21 | | | 0.12 | 4.5 | 32 | 3.85 | | | |
| | 0.06 | 17 | 64 | 3.85 | | | 0.062 | 18.4 | 64 | 4 | | | |
| Tetramer | 0.468 | 10.2 | 8 | 3.741 | 3.37 | 7.93 | 0.475 | 3.2 | 8 | 3.802 | 3.54 | 3.33 | 1.05 |
| M00-127 | 0.221 | 5.4 | 16 | 3.54 | | | 0.223 | 2.4 | 16 | 3.57 | | | |
| | 0.089 | 8.2 | 32 | 2.83 | | | 0.101 | 4.4 | 32 | 3.24 | | | |
| | 0.065 | 8.8 | 64 | 4.16 | | | 0.067 | 2.7 | 64 | 4.27 | | | |
| Tetramer | 0.724 | 2.4 | 8 | 5.79 | 5.68 | 7.1 | 0.714 | 1.5 | 8 | 5.71 | 5.77 | 3.87 | 1.02 |
| M00-130 | 0.361 | 12.8 | 16 | 5.77 | | | 0.375 | 5.2 | 16 | 6.00 | | | |
| | 0.171 | 6.1 | 32 | 5.48 | | | 0.175 | 4.9 | 32 | 5.6 | | | |
| | 0.068 | 5.1 | 64 | 4.33 | | | 0.074 | 3.8 | 64 | 4.76 | | | |
| Mean | | | | | | 8.10 | | | | | | 4.13 | |

TABLE XXIII
Summary of dilution experiments

| | Group | | | | | | | | | | | | |
|---|---|---|---|---|---|---|---|---|---|---|---|---|---|
| | | I | | II | | III | | IV | | V | | VI | |
| | | D-00-031 | | D-00-032 | | D-00-032 | | D-00-033 | | D-00-031 | | D-00-033 | |
| | | Mean | | Mean | | Mean | | Mean | | Mean | | Mean | % |
| | Sample | (μg/ml) | % CV | (μg/ml) | % CV | (μg/ml) | % CV | (μg/ml) | % CV | (μg/ml) | % CV | (μg/ml) | CV |
| Monomer | M-00-044 | 8 | 6.9 | 7 | 4.5 | 8.5 | 11.7 | 9 | 13.7 | 8.58 | 7.47 | 8.43 | 5.77 |
| Monomer | M-00-045 | 5.4 | 5.9 | 4.8 | 8.8 | 5.9 | 6 | 6.4 | 3.7 | 5.83 | 9 | 6.03 | 5.83 |
| Monomer | M-00-102 | 7.4 | 11.3 | 6.9 | 7.1 | 9 | 2.4 | 9.4 | 4.9 | 8.28 | 5.4 | 8.24 | 2.63 |
| Tetramer | M-00-124 | 4.2 | 3.3 | 3.9 | 10.2 | 4.3 | 3.3 | 4.4 | 5.2 | 3.92 | 11.7 | 4.03 | 3.3 |
| Tetramer | M-00-127 | 3.9 | 8.4 | 3.8 | 2.4 | 3.8 | 3.4 | 4.1 | 4 | 3.37 | 7.93 | 3.54 | 3.33 |
| Tetramer | M-00-130 | 6.4 | 3.2 | 5.8 | 6.9 | 6.4 | 5.9 | 6.4 | 2 | 5.68 | 7.1 | 5.77 | 3.87 |
| Mean | | | 6.5 | | 6.65 | | 5.45 | | 5.6 | | 8.10 | | 4.12 |

The mean of the % CV for all data was calculated to be =6.07 (for n=6), which is excellent precision. In order to calculate the variability intra plates, the value obtained with two different plates for the same sample was calculated. Table XXIV summarizes these ratios. A very good correlation was observed between the plates tested the same day. The global ratio was found to be: 0.999±0.059 for n=18 with a % CV of 5.98.

TABLE XXIV
Calculated ratio

| | | Ratio | | |
|---|---|---|---|---|
| | | | | Day 3 |
| | | Day 1 | Day 2 | D00-031/ |
| | Sample | D00-031/D00-032 | D00-032/D00-033 | D00-033 |
| Monomer | M-00-044 | 0.88 | 1.05 | 0.98 |
| Monomer | M-00-045 | 0.88 | 1.09 | 1.03 |
| Monomer | M-00-102 | 0.93 | 1.04 | 1.00 |
| Tetramer | M-00-124 | 0.93 | 1.03 | 1.03 |
| Tetramer | M-00-127 | 0.98 | 1.06 | 1.05 |
| Tetramer | M-00-130 | 1.01 | 1.00 | 1.02 |
| Mean | | 0.935 | 1.045 | 1.018 |
| SD | | 0.052 | 0.030 | 0.025 |
| % CV | | 5.609 | 2.887 | 2.439 |

Summary of all ratios

| | |
|---|---|
| Mean | 0.999 |
| SD | 0.059 |
| N | 18 |
| % CV | 5.98 |

TABLE XXV
Statistical analysis

| Group | I | II | III | IV | V | VI |
|---|---|---|---|---|---|---|
| Operator | A | A | B | B | C | C |
| Day | 1 | 1 | 2 | 2 | 3 | 3 |
| Plate | D00-031 | D00-032 | D00-032 | D00-033 | D00-031 | D00-033 |
| Temperature | 21° C. | 21° C. | 28° C. | 28° C. | 31° C. | 31° C. |
| Reader | 1 | 1 | 2 | 2 | 3 | 3 |

| | | | | | | | Statistical analysis | | | | | |
|---|---|---|---|---|---|---|---|---|---|---|---|---|
| | | | | | | | Coch- | | | | | |
| | Variance | Variance | Variance | Variance | Variance | Variance | ran | Sr2 | Sg2 | SR2 | CV rep | CV repro |
| Monomer | 0.307 | 0.102 | 1 | 1.49 | 0.373 | 0.244 | 0.424 | 0.586 | 0 | 0.586 | 9% | 9.3% |
| M00-044 | | | | | | | | | | | | |
| n | 3 | 3 | 3 | 3 | 3 | 3 | | | | | | |
| Monomer | 0.102 | 0.179 | 0.126 | 0.055 | 0.270 | 0.032 | 0.353 | 0.127 | 0.23 | 0.356 | 6% | 10.4% |
| M00-045 | | | | | | | | | | | | |
| N | 3 | 3 | 3 | 3 | 3 | 3 | | | | | | |
| Monomer | 0.710 | 0.239 | 0.046 | 0.212 | 0.423 | 0.069 | 0.418 | 0.283 | 0.65 | 0.936 | 6% | 11.8% |
| M00-102 | | | | | | | | | | | | |
| N | 3 | 3 | 3 | 3 | 3 | 3 | | | | | | |
| Tetramer | 0.019 | 0.162 | 0.021 | 0.053 | 0.071 | 0.074 | 0.405 | 0.067 | 0.03 | 0.093 | 6% | 7.4% |
| M00-124 | | | | | | | | | | | | |
| N | 3 | 3 | 3 | 3 | 3 | 3 | | | | | | |
| Tetramer | 0.105 | 0.009 | 0.017 | 0.026 | 0.229 | 0.080 | 0.491 | 0.078 | 0.04 | 0.115 | 7% | 9.1% |
| M00-127 | | | | | | | | | | | | |
| N | 3 | 3 | 3 | 3 | 3 | 3 | | | | | | |
| Tetramer | 0.041 | 0.162 | 0.144 | 0.017 | 0.030 | 0.043 | 0.371 | 0.073 | 0.09 | 0.160 | 4% | 6.6% |
| M00-130 | | | | | | | | | | | | |
| n | 3 | 3 | 3 | 3 | 3 | 3 | | | | | | |
| Mean | | | | | | | | | | | 6.69% | 9.1% |

C(0.05, 3, 6) 0.616;
C(0.01, 3, 6) 0.722

TABLE XXVI
Summary of spiking experiments results and statistical analysis

| Group | | | | |
|---|---|---|---|---|
| I | II | III | IV | VI |
| Operator | | | | |
| A | A | B | B | C |
| Day | | | | |
| 1 | 1 | 2 | 2 | 3 |
| Plate | | | | |
| D00-031 | D00-032 | D00-032 | D00-033 | D00-033 |
| Temperature | | | | |
| 21° C. | 21° C. | 28° C. | 28° C. | 31° C. |
| Reader | | | | |
| 1 | 1 | 2 | 2 | 3 |

| | % of | | | % of | | | % of | | | % of | | | % | | |
|---|---|---|---|---|---|---|---|---|---|---|---|---|---|---|---|
| Sample | recovery | SD | % CV | recovery | SD | % CV | recovery | SD | % CV | recovery | SD | % CV | of recovery | SD | % CV |
| Monomer | 80.4 | 7.6 | 9.4% | 81.9 | 0 | 0% | 89.1 | 0 | 0.0% | 92.3 | 0 | 0.0% | 100.7 | 4 | 4.4% |
| M00-044 | | | | | | | | | | | | | | | |
| Monomer | 87.2 | 1.8 | 2.0% | 87.3 | 5.2 | 6% | 75 | 3.2 | 4.3% | 80.6 | 4.6 | 5.7% | 92.3 | 4 | 4.6% |
| M00-045 | | | | | | | | | | | | | | | |
| Monomer | 86.8 | 4 | 4.6% | 93.5 | 4.6 | 5% | 85.4 | 1.8 | 2.0% | 93.6 | 3.8 | 4.0% | 109 | 9 | 8.6% |
| M00-102 | | | | | | | | | | | | | | | |
| Tetramer | 92 | 7.7 | 8.3% | 91 | 12.1 | 13% | 85.6 | 6.9 | 8.1% | 90.1 | 2.1 | 2.4% | 103.8 | 11 | 10.4% |
| M00-124 | | | | | | | | | | | | | | | |
| Tetramer | 86.8 | 4.4 | 5.1% | 86.1 | 3 | 3% | 85.8 | 5.3 | 6.1% | | 8.6 | 7.0% | 111.8 | 17 | 15.2% |
| M00-127 | | | | | | | | | | | | | | | |
| Tetramer | 75.1 | 12.7 | 17% | 79 | 11.4 | 14% | 78.3 | 5.8 | 7.4% | 89.2 | 2.1 | 2.3% | 104.9 | 13 | 12.8% |
| M00-130 | | | | | | | | | | | | | | | |
| Variance | 35.801 | | | 29.490 | | | 28.684 | | | 25.933 | | | 46.819 | | |
| Mean | 84.72 | | | 86.47 | | | 83.20 | | | 89.16 | | | 103.75 | | |
| SD | 5.98 | | | 5.43 | | | 5.36 | | | 5.09 | | | 6.84 | | |
| CV | 7.06 | | | 6.28 | | | 6.44 | | | 5.71 | | | 6.60 | | |

The sensitivity was calculated by measuring the “zero” standard twenty times. The mean and the SD were calculated. From these values, the mean-3SD was calculated, and the value obtained was interpolated into corresponding standard curve. Results are shown in Table XXVII. The mean of the sensitivity was 0.0405 μg/ml.

TABLE XXVII
Summary of sensitivity values

| Group | I | II | III | IV | V | VI |
|---|---|---|---|---|---|---|
| Mean | 0.674 | 0.584 | 0.513 | 0.613 | 0.832 | 0.868 |
| SD | 0.014 | 0.0121 | 0.0056 | 0.021 | 0.0385 | 0.0293 |
| CV | 2.1 | 2.1 | 1.09 | 3.43 | 4.63 | 3.38 |
| N | 20 | 20 | 20 | 20 | 20 | 20 |
| Mean-3SD | 0.632 | 0.548 | 0.514 | 0.548 | 0.717 | 0.780 |
| Calculated value | 0.03 | 0.02 | 0.011 | 0.056 | 0.074 | 0.052 |

Comparison of Standard Curves

Six different standard curves were determined at three different temperatures (21 ° C., 28° C., and 31° C.), each during one day of experiments. To compare the different standard curves, the optical densities were normalized by calculating the B/Bmax (B is the signal obtained for each standard point; Bmax is the signal obtained with the standard zero). Results are summarized in Table XXVIII.

TABLE XXVIII
Summary of standard curves

| | Temperature 21° C. | | | 28° C. | | | 31° C. | | |
|---|---|---|---|---|---|---|---|---|---|
| μg/ml b2m | Mean | SD | CV | Mean | SD | CV | Mean | SD | CV |
| 1 | 17.27 | 0.93 | 5.38 | 12.52 | 0.89 | 7.14 | 15.79 | 1.41 | 8.91 |
| 0.5 | 21.93 | 1.04 | 4.75 | 19.04 | 0.87 | 4.57 | 21.44 | 0.83 | 3.87 |
| 0.25 | 38.12 | 3.80 | 9.97 | 38.96 | 3.78 | 9.70 | 38.71 | 3.59 | 9.28 |
| 0.125 | 62.57 | 4.87 | 7.78 | 67.70 | 4.25 | 6.28 | 65.79 | 1.69 | 2.57 |
| 0.0625 | 80.68 | 3.30 | 4.08 | 84.12 | 2.24 | 2.66 | 81.70 | 4.25 | 5.20 |
| 0.03125 | 90.38 | 3.20 | 3.54 | 93.66 | 1.51 | 1.61 | 90.66 | 5.38 | 5.94 |
| 0.0156 | 94.82 | 2.19 | 2.31 | 96.75 | 1.78 | 1.83 | 96.56 | 3.59 | 3.72 |
| 0.0 | 100.00 | 0.00 | 0.00 | 100.00 | 0.00 | 0.00 | 99.84 | 0.39 | 0.39 |

All of the standard curves overlapped. The results indicate that the temperature has no influence on the slope of the curves, and also has no influence on the final result.

Correlation of Free β2-microglobulin Detected Using EIA and SEC

The content in free β2-microglobulin for all monomer and tetramer samples was analyzed by EIA and compared to that determined by SEC (size exclusion chromatography; Table XXIX). The comparison was performed taking into account the concentration obtained from dilution experiments.

TABLE XXIX
Values obtained with EIA and SEC for all samples tested.

| | Sample | EIA | SEC |
|---|---|---|---|
| Monomer | M-00-044 | 8.252 | 7.11 |
| Monomer | M-00-045 | 5.727 | 4.5 |
| Monomer | M-00-102 | 8.203 | 7.29 |
| Tetramer | M-00-124 | 4.125 | 3.97 |
| Tetramer | M-00-127 | 3.752 | 3.57 |
| Tetramer | M-00-130 | 6.075 | 6.13 |

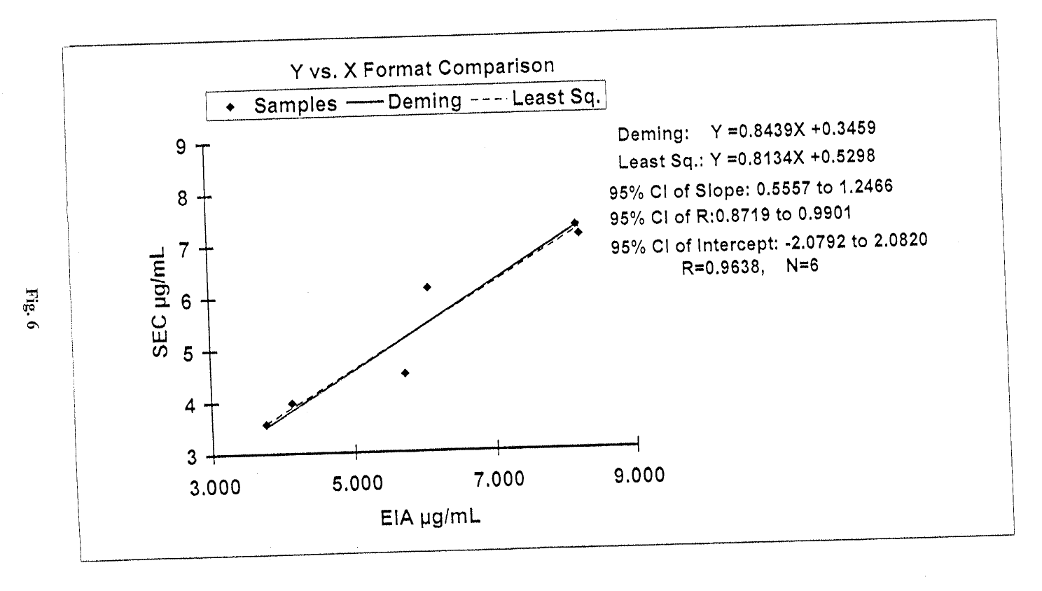

The data was analyzed by least squares and Deming linear regression. Results are shown in FIG. 6 and summarized below.

The correlation parameters between EIA and SEC were:

| Parameters | Least Square | Deming |
|---|---|---|
| Correlation Coefficient r | 0.929 | 0.9638 |
| Sample size | 6 | 6 |
| 95 confidence interval for r | 0.6990 to 0.9962 | 0.8719 to 0.9901 |

Conclusion

These results demonstrate the EIA assay was robust, accurate, reproducible and sensitive. The three different lots of plates were homogenous as demonstrated by the low CV (<10%). The calculated values passed the acceptance criteria defined in the validation protocol. The Tables below summarize the criteria established and the values found.

| Test | Criteria | Precision CV | Reproducibility CV |
|---|---|---|---|
| Dilution experiments | <10% CV | 6.69 | 9.1 |
| Repeatability | <10% CV | 8.36 | 9.67 |
| Spiked experiments | <10% CV | 5.89 | 7.5 |

| Test | Criteria | Calculated |
|---|---|---|
| Recovery | >80% | 93.1 |
| Sensitivity | 0.05 ug/ml | 0.0405 ug/ml |

| | Criteria | Calculated |
|---|---|---|
| Correlation Coefficient r | >0.90 | 0.9638 |
| Sample size | | 6 |
| P = | | 0.0021 |
| 95 confidence interval for r | | 0.6990 to 0.9962 |

Although the invention has been described with reference to the above examples, it will be understood that modifications and variations are encompassed within the spirit and scope of the invention. Accordingly, the invention is limited only by the following claims.

What is claimed is:

1. A method of detecting the presence of free β2-microglobulin in a sample containing complexes comprising β2-microglobulin and a β2-microglobulin associated protein (β2m-AP), the method comprising
a) contacting the sample with an immobilized antibody, or antigen binding fragment thereof, that specifically binds β2-microglobulin, but does not substantially bind a β2-microglobulin/β2m-AP complex, under conditions that allow specific binding of the immobilized antibody, or antigen binding fragment thereof, and β2-microglobulin; and
b) detecting specific binding of the immobilized antibody to β2-microglobulin, thereby detecting the presence of free β2-microglobulin in the sample.

2. The method of claim 1, wherein the β2-microglobulin is a human β2-microglobulin or a murine β2-microglobulin.

3. The method of claim 1, wherein the β2m-AP is a major histocompatibility complex (MHC) class I molecule.

4. The method of claim 3, wherein the MHC class I molecule is a human lymphocyte antigen (HLA) molecule or a murine H2 molecule.

5. The method of claim 4, wherein the HLA molecule is an HLA-A, HLA-B or HLA-C molecule.

6. The method of claim 4, wherein the murine H2 molecule is an H2-D, H2-K or H2-L molecule.

7. The method of claim 3, wherein the MHC class I molecule is an MHC class Ib molecule.

8. The method of claim 7, wherein the MHC class Ib molecule is an HLA-E, HLA-F or HLA-G molecule.

9. The method of claim 1, wherein the β2m-AP is a hemochromatosis gene product, HFE, which is involved in iron metabolism.

10. The method of claim 1, wherein the β2m-AP is a cluster of differentiation (CD) molecule selected from CD1a, CD1b, CD1dc CD1d and CD1e.

11. The method of claim 1, wherein the immobilized antibody is C21.48A or an antibody having substantially the same specific binding activity of C21.48A.

12. The method of claim 1, wherein detecting specific binding of the immobilized antibody to β2-microglobulin comprises
further contacting the sample and the immobilized antibody, or antigen binding fragment thereof, with a second antibody that specifically binds β2-microglobulin, including β2-microglobulin that is specifically bound to an immobilized antibody, under conditions that allow specific binding of the second antibody;
isolating the immobilized antibody, including β2-microglobulin specifically bound to said immobilized antibody and second antibody specifically bound to said β2-microglobulin, thereby obtaining isolated immobilized antibody; and
detecting second antibody associated with the isolated immobilized antibody, thereby detecting the presence of free β2-microglobulin in the sample.

13. The method of claim 12, wherein the second antibody specifically binds free β2-microglobulin, a β2-microglobulin/β2m-AP complex, or free β2-microglobulin and a β2-microglobulin/β2m-AP complex.

14. The method of claim 12, wherein the second antibody is B1G6 or an antibody having substantially the same specific binding activity of B1G6.

15. The method of claim 12, wherein the second antibody comprises a detectable label, and wherein said detecting second antibody comprises detecting the detectable label.

16. The method of claim 12, wherein said detecting second antibody associated with immobilized antibody comprises contacting the second antibody with a reagent that specifically binds to the second antibody, under conditions that allow specific binding of the reagent to the second antibody, and detecting specific binding of the reagent to the second antibody.

17. The method of claim 16, wherein the reagent is a third antibody.

18. The method of claim 1, wherein detecting specific binding of the immobilized antibody to β2-microglobulin comprises
isolating the immobilized antibody, including β2-microglobulin specifically bound to said immobilized antibody, from material not specifically bound to the immobilized antibody, thereby obtaining isolated immobilized antibody;
further contacting the isolated immobilized antibody, with a second antibody that specifically binds β2-microglobulin, including β2-microglobulin that is specifically bound to an immobilized antibody, under conditions that allow specific binding of the second antibody; and
detecting second antibody associated with the isolated immobilized antibody, thereby detecting the presence of free β2-microglobulin in the sample.

19. The method of claim 1, wherein contacting the sample and the immobilized antibody, or antigen binding fragment thereof, further comprises contacting the sample and immobilized antibody, or antigen binding fragment thereof, with competitor β2-microglobulin; and
wherein detecting specific binding of the immobilized antibody to β2-microglobulin comprises detecting competitor β2-microglobulin specifically bound to the immobilized antibody, which is indicative of free β2-microglobulin in the sample, thereby detecting the presence of free β2-microglobulin in the sample.

20. The method of claim 19, wherein the competitor β2-microglobulin comprises a detectable label.

21. The method of claim 20, wherein the detectable label comprises an enzyme, a fluorescent molecule, a luminescent molecule, or a radionuclide.

22. The method of claim 20, wherein the detectable label comprises alkaline phosphatase.

22. A method of detecting the presence of β2-microglobulin, which is not bound to a major histocompatibility complex (MHC) class I molecule, in a sample containing β2-microglobulin/MHC class I molecule complexes, the method comprising
a) contacting the sample with an immobilized antibody, or antigen binding fragment thereof, that specifically binds β2-microglobulin, but does not substantially bind a β2-microglobulin/MHC molecule complex, under conditions that allow specific binding of the antibody, or antigen binding fragment thereof, and β2-microglobulin; and
b) detecting specific binding of the antibody to β2-microglobulin, thereby detecting the presence of β2-microglobulin that is not bound to an MHC molecule in the sample.

23. The method of claim 22, wherein the β2-microglobulin is a human β2-microglobulin or a murine β2-microglobulin.

24. The method of claim 22, wherein the MHC class I molecule is a human lymphocyte antigen (HLA) molecule.

25. The method of claim 24, wherein the HLA molecule is an HLA-A, HLA-B or HLA-C molecule.

26. The method of claim 22, wherein the MHC class I molecule is a murine H2 molecule.

27. The method of claim 26, wherein the murine H2 molecule is an H2-D, H2-K or H2-L molecule.

28. The method of claim 22, wherein β2-microglobulin/MHC class I molecule complexes in the sample comprise
β2-microglobulin/MHC class I molecule monomers, wherein a monomer comprises one β2-microglobulin and one MHC class I molecule;
β2-microglobulin/MHC class I molecule polymers, wherein each polymer comprises at least two operatively linked β2-microglobulin/MHC class I molecule monomers; or
a combination of β2-microglobulin/MHC class I molecule monomers and β2-microglobulin/MHC class I molecule polymers.

29. The method of claim 28, wherein the β2-microglobulin/MHC class I molecule polymer comprises a tetramer.

30. The method of claim 22, wherein the MHC class I molecule further comprises a linker moiety.

31. The method of claim 30, wherein the β2-microglobulin/MHC class I molecule complexes in a sample comprise β2-microglobulin/MHC class I molecule polymers, and wherein each monomer in a polymer is operatively linked to at least one other monomer through the linker moiety.

32. The method of claim 31, wherein the linker moiety comprises a thiol reactive group, and wherein the monomers are operatively linked through a disulfide bond.

33. The method of claim 30, wherein the linker moiety comprises a first member of a specific binding pair, which interacts specifically with a second member of the specific binding pair.

34. The method of claim 33, wherein the β2-microglobulin/MHC class I molecule complexes in a sample comprise β2-microglobulin/MHC class I molecule polymers, and wherein each monomer in a polymer is operatively linked through a specific interaction of the first member of a specific binding pair and a second member of the specific binding pair.

35. The method of claim 33, wherein the specific binding pair comprises biotin and avidin, or biotin and streptavidin.

36. The method of claim 22, wherein the MHC class I molecule in the β2-microglobulin/MHC class I molecule complex comprises a peptide antigen binding domain.

37. The method of claim 36, wherein the sample further comprises a peptide that can bind to the peptide antigen binding domain in the MHC class I molecule.

38. A kit, comprising
a) an antibody, or antigen binding fragment thereof, which specifically binds free β2-microglobulin,
wherein said antibody, or antigen binding fragment thereof, does not substantially bind a β2-microglobulin/MHC class I molecule complex, and
wherein said antibody, or antigen binding fragment thereof is immobilized on a solid support; and
b) competitor β2-microglobulin, which can be specifically bound by the antibody, or antigen binding fragment thereof.

39. The kit of claim 38, wherein the immobilized antibody is C21.48A or an antibody having substantially the same specific binding activity of C21.48A.

40. The kit of claim 38, wherein the competitor β2-microglobulin comprises a detectable label.

41. The kit of claim 40, wherein the detectable label comprises an enzyme, a fluorescent molecule, a luminescent molecule, or a radionuclide.

42. The kit of claim 41, wherein the detectable label comprises alkaline phosphatase.

43. The kit of claim 38, further comprising at least one standard, which comprises a predetermined amount or concentration of free β2-microglobulin.

44. A kit, comprising
a) a first antibody, or antigen binding fragment thereof, which specifically binds free β2-microglobulin, wherein said first antibody, or antigen binding fragment thereof, does not substantially bind a β2-microglobulin/MHC class I molecule complex; and
b) a second antibody, or antigen binding fragment thereof, which specifically binds free β2-microglobulin, a β2-microglobulin/MHC class I molecule complex, a complex comprising β2-microglobulin and the first antibody, or a combination thereof.

45. The kit of claim 44, wherein said first antibody or antigen binding fragment thereof, is immobilized to a solid support.

46. The kit of claim 44, wherein the first antibody is C21.48A or an antibody having substantially the same specific binding activity of C21.48A.

47. The method of claim 44, wherein the second antibody is B1G6 or an antibody having substantially the same specific binding activity of B1G6.

48. The kit of claim 44, further comprising free β2-microglobulin, a β2-microglobulin/MHC class I molecule complex, or a combination thereof.

49. The kit of claim 48, wherein the free β2-microglobulin comprises a standard, which comprises a predetermined amount or concentration of free β2-microglobulin.

50. The kit of claim 49, wherein the standard comprises one of a plurality of standards, the plurality comprising at least two different amounts or concentrations of free β2-microglobulin.

51. The kit of claim 44, wherein the second antibody, or antigen binding fragment thereof, comprises a detectable label.

52. The kit of claim 51, wherein the detectable label comprises an enzyme, a fluorescent molecule, a luminescent molecule, or a radionuclide.

53. The kit of claim 51, wherein the detectable label comprises a peroxidase.

54. The kit of claim 44, further comprising a third antibody, which specifically binds the second antibody.
